(19) United States
(12) Patent Application Publication
(10) Pub. No.: US 2003/0138490 A1
(43) Pub. Date: Jul. 24, 2003

(54) Synthesis and uses of polymer gel nanoparticle networks

(76) Inventors: Zhibing Hu, Dallas, TX (US); Xihua Lu, Dallas, TX (US); Jun Gao, Dallas, TX (US); Bill C. Ponder, Gropevine, TX (US); John St. John, Gropevine, TX (US); Daniel G. Moro, Dallas, TX (US)

Correspondence Address: JACKSON WALKER, L.L.P., SUITE 2100, 112 EAST PECAN ST., SAN ANTONIO, TX, 78205, US

(21) Appl. No.: 10/215,564

(22) Filed: Aug. 9, 2002

Related U.S. Application Data
(60) Provisional application No. 60/311,036, filed on Aug. 9, 2001.

(51) Int. Cl. A61K 9/14

(52) U.S. Cl. 424/486; 424/488

(57) ABSTRACT

Disclosed is a new class of nanostructured polymeric materials comprising polymer gel nanoparticles that are covalently bonded through functional groups on the surfaces of neighboring particles. These nanoparticles may be prepared as suspensions in an aqueous or, non-aqueous environment. These gels have two unique and different structural networks; the primary network comprises crosslinked polymer chains in each individual particle, while the secondary network is a system of crosslinked nanoparticles. Particular polymer gel nanoparticle network compositions disclosed herein may function as carriers for controlled delivery of pharmaceuticals or other chemical agents, gel sensors and other commercial applications.

The present application claims priority to U.S. Provisional Patent Application Serial No. 60/311,036, filed Aug. 9, 2001, the entire contents of which specifically incorporated herein by reference in its entirety without disclaimer. The United States government has certain rights in the present invention pursuant to Grant DAAG55-98-1-0175 from the United States Army Research Office.

### BACKGROUND OF THE INVENTION

1. Field of the Invention

The present invention relates to nanostructured, polymeric gel materials, and in particular to matricies and nanoparticle networks comprising novel nanoparticle compositions. Also provided are methods for the synthesis and use of such compounds in the formulation of pharmaceutical compounds, and in the preparation of medicaments for use in therapy.

2. Description of Related Art

Hydrogels are three-dimensional macromolecular networks that contain a large fraction of water within their structure and do not dissolve. These materials exhibit high water content and are soft and pliable. These properties are similar to natural tissue, and therefore hydrogels are very biocompatible and are particularly useful in biomedical and pharmaceutical applications.

Hydrogels usually respond to a variety of external, environmental conditions. Some can reversibly swell or shrink up to 1000 times in volume based upon changes in pH and temperature, for example. These unique properties and other characteristics are thoroughly detailed in the scientific reference articles cited above.

Some diversified uses of responsive gels include solute/solvent separations, biomedical applications, controlled drug delivery, sensors and devices, and in NMR contrast agents. These applications are described in U.S. Pat. Nos. 4,555,344, 4,912,032, 5,062,841, 5,976,648 and 5,532,006, respectively (each of which is specifically incorporated herein by reference in its entirety without disclaimer).

Polymer gels can be formed by the free radical polymerization of monomers in the presence of a reactive crosslinking agent and a solvent. They can be made either in bulk or in nano- or micro-particle form. The bulk gels are easy to handle, but usually have very slow swelling rates and amorphous structures arising from randomly crosslinked polymer chains. However, gel nanoparticles react quickly to an external stimulus, but may be too small for some practical applications.

Responsive polymer gels can be made by the co-polymerization of two different monomers, by producing interpenetrating polymer networks or by creating networks with microporous structures. These processes are described in U.S. Pat. Nos. 4,732,930, 5,403,893, and 6,030,442 (each of which is specifically incorporated herein by reference in its entirety without disclaimer). In U.S. Pat. No. 6,187,599, polymer gels were also used to embed self-assembled colloidal polymer solid spheres. Finally, a microparticle composition and its method of use in drug delivery and diagnostic applications have also been described in U.S. Pat. No. 5,654,006 (this and the prior patent are incorporated by reference herein).

In the present invention, by first making gel nanoparticles and then bonding them together, a new class of gels with two levels of structural difference has been engineered; the primary network and the secondary network. As shown by a conceptual model in FIG. 1A and FIG. 1B, there are two different networks in a gel nanoparticle network. The primary network comprises of crosslinked polymer chains inside each nanoparticle, while the secondary network comprises nanoparticles crosslinked with each other. This secondary configuration is depicted in the optical microscopic image of a hydroxypropylcellulose (HPC) nanoparticle network in water at room temperature shown in FIG. 1C.

The mesh size (the average distance between two neighboring crosslinkers) of the primary network depends on the concentration ratio of the crosslinker to linear polymer chains or monomers and is usually around 1-10 nm. In comparison, the mesh size of the secondary network depends on the concentration and type of the crosslinker and the concentration and size of the nanoparticles. The mesh size of this secondary network is typically around 50-500 nm. As a result, the nanoparticle network could be used to entrap and deliver small actives and/or very large biomolecules within its primary and secondary structures. This unique attribute will enhance the versatility of polymer gel nanoparticle networks as potential carriers to provide controlled delivery of a variety of active compounds.

Such nanostructured gels have unique and useful properties that conventional gels do not have, including, for example, a high surface area, a unique and distinguishable color at room temperature, and the ability to be easily combined if desired to yield heterogeneous networks consisting of diversified physical and chemical properties. The compositions and methods of the present invention provide useful improvements in a variety of technological applications, including, for example, controlled delivery of drugs or other actives, optical and calorimetric sensors, interferometer systems, holographic or interference gratings, integrated circuit lithography, optical displays, environmental cleanup agents and bio-adhesives.

In the present invention, several of the polymer nanoparticle gels are prepared using degradable crosslinkers. Using these particles as building blocks, the degradable aspects of the nanoparticle networks have been used to affect controlled drug release. The drug release rate will depend on both drug molecular diffusion, strongly influenced by network pore-size, and the degradation rate of the crosslinkers.

### SUMMARY OF THE INVENTION

The present invention overcomes limitations in the prior art by providing a new class of nanostructured polymer gels and methods for their synthesis by crosslinking gel nanoparticles dispersed in an aqueous or non-aqueous medium through covalent bonds between functional groups on the surfaces of neighboring particles. These gels have two levels of structural configuration; a primary network consisting of crosslinked polymer chains in each nanoparticle, and a secondary network composed of nanoparticles crosslinked together as a whole. With such a unique composite structure, these networks have new properties that conventional gels do not have, including a high surface area, a distinguishable and unique color at room temperature and a uniform and easily regulated mesh size.

The method of manufacture comprises synthesizing polymer gel nanoparticles, self-assembling them into a 3D network, and eventually covalently bonding them together. The covalent bonding contributes to the structural stability, while self-assembly provides structures that could diffract light in addition to other unique physical properties. The polymer gel nanoparticle network exhibits controlled changes in volume in response to external environmental changes. The incorporation of biodegradable crosslinkers into either the polymer gel nanoparticles or between the nanoparticles provide networks that exhibit degradable properties.

Various architectures of nanostructured gels can be easily tailored by selecting different gel nanoparticles and crosslinking agents. Such stable three-dimensional structures provide a diversified functionality not only from the constituent gel building blocks but also from the long-range ordering that characterizes these structures. It is desirable to develop and produce new polymer gels that exhibit predictable and reversible characteristics in response to external environmental changes. Several potential applications of gel nanoparticle networks are disclosed in this application, including a nanoparticle network with a fast shrinking rate, a light-scattering colored nanoparticle network, and a co-nanoparticle network as a potential multi-functional drug delivery carrier.

In one embodiment, the invention provides a composition comprising the nanostructured polymeric networks and materials described herein. The composition may be formulated for use in a variety of environmental, industrial, and medical applications, including, for example, detoxification and entrapment of various chemicals, ions, metals, and radioactive and/or chemical wastes, such as for example, in various bioremediation applications. The compositions disclosed herein may also be formulated for use in adhesives, and in particular, bioadhesives, owing to the mucoadhesive properties various of the polymeric nanoparticle networks possess. Particularly preferred bioadhesive materials include nanoparticles that comprise at least a first polymer selected from the group consisting of HPC, NIPA, PVA, PPO, PEO, PPO copolymer, and PEO.

In some embodiments, the pluralities of nanoparticles, nanoparticle networks and nanostructured polymeric matrices me be formulated comprising one or more pharmaceutical excipient, diluents, buffers, and such like as may be for administration of the active compounds to an animal, such as administration to human and non-human mammals under the care of a medical provider, such as a physician, dentist, or in the case of non-human mammals, a licensed veterinarian or veterinary practicioner.

In related embodiments, the invention provides a controlled-release, sustained-release, time-release, or delayed-release pharmaceutical delivery system. These systems typically comprise one or more of the compositions disclosed herein and at least a first diagnostic, therapeutic, or prophylactic medicament. Such medicaments may be formulated for oral, intravenous, intraarterial, intradermal, subcutaneous, sublingual, inhalation, transdermal, intrathecal, intraossius, intranasal, intraocular, or intracellular administration, as may be required by the particular use regimen in which the system is employed.

Likewise, the invention also provides diagnostic, prophylactic, and therapeutic kits that comprise one or more of the disclosed nanostructured polymeric materials. These kits may optionally comprise additional therapies, reagents, buffers, diluents, etc. and will typically also include instructions for using the kit in the particular applications for which it has been designed. These kits may contain at least a first peptide, polypeptide, protein, vaccine, antisense oligonucleotide, hormone, growth factor, polynucleotide, vector, ribozyme, or at least a first diagnostic, therapeutic, or prophylactic medicament.

The invention also provides methods of controlling the delivery of a pharmaceutical compound to a target site on, or within the body of an animal, with these methods generally involving administration to the animal a biologically-effective amount of the controlled-release pharmaceutical delivery system, for a time effective to deliver the particular compound(s) associated with, or entrapped within, the polymeric nanoparticle matrix of the system.

When desirable, the disclosed compositions may be used to delay or sustain the delivery of a pharmaceutical compound to a first target site of a mammal. This method typically involves providing to, or administering to the selected human patient or mammal, a biologically-effective amount of the controlled-release pharmaceutical delivery systems disclosed herein effective to delay or sustain the delivery of one or more therapeutic compounds associated with, or entrapped within the system. Such methods are particularly desirable when the selected target site is a cell, tissue, gland, bone, tumor, or an organ within the body of a mammal. Using the nanoparticle networks, it is possible to delay the diffusion of the active compounds, so that the drug may be provided well after the initial administration is made to the animal. (For example, long-term therapy following a single injection of the controlled release formulation).

In such instances, the compound may be delivered to the target site within a period of from about 10 min or less to about 24 hrs or more following administration of the pharmaceutical delivery system to the mammal. When relatively rapid delivery is required, the networks may be selected to provide the compound to the target site within a period of about 10, 15, 20, 25, 30, 35, 40, 450, 50, 55, or 60 min or more following administration of the pharmaceutical delivery system that contains the therapeutic compound to the mammal.

When relatively slower delivery is required, the networks may be selected to provide the compound to the target site within a period of about 1 hr, 2 hr, 3 hr, 4 hr, 5 hr, 6 hr, 7 hr, or even 8 hr or more following administration of the pharmaceutical delivery system that contains the therapeutic compound to the mammal.

In other embodiments, it may be desirable to have significantly longer sustained release of the active compounds to the target site. In such cases, the networks may be fabricated to provide release of the active ingredients to the target site within a period of about 10, 12, 14, 16, 18, 20, 22, or 24 hrs or more, and even longer times such as sustained delivery of a target compound for a period of 2, 3, 4, 5, 6, 7, 8, 9, 10, 14, 21, 30, 60, or 90 days or more following administration of the pharmaceutical delivery system that contains the therapeutic compound to the mammal.

The invention also provides methods of remediating toxic wastes, and decontaminating radioactively-, chemically- or biologically-contaminated sites. These methods generally involve applying to, providing to, or contacting the site with one or more applications of remediation-effective amounts of the disclosed nanostructured polymeric networks for a time period effective to alter, reduce, remove, or remediate the contaminants from the particular site to which the compounds have been applied. Preferred sites include environmental, commercial, residential or industrial sites, as well as the site of an industrial accident, motor vehicle accident, chemical spill, and such like. The method may be used for radioactive, chemical, or biological contaminant, and in such embodiments, the nanoparticle network that comprises at least a first functionalized moiety, or a free ionic charge on one or more surfaces of the nanoparticles or the nanoparticle network.

The invention also provides methods for preparing the nanostructured polymeric gels and matrices disclosed herein. These methods typically comprise the steps of:

(a) contacting a plurality of polymeric gel nanoparticles under conditions effective to permit self-assembly of a substantial population of the polymeric gel nanoparticles into a network of nanoparticles; and

(b) reacting said network of nanoparticles with at least a first cross-linking agent under conditions effective to substantially covalently crosslink the network of nanoparticles to produce the desired nanostructured polymeric gel.

The crosslinking agent may be a degradable crosslinking agent, such as a biodegradable crosslinking agent, such as divinyl sulfone. The polymeric gel nanoparticles may be comprised of HPC, NIPA, PVA, PPO, PEO, PPO copolymer, or PEO copolymer nanoparticles.

The plurality of polymeric gel nanoparticles may comprise a population of internally-crosslinked nanoparticles, or a population of colloidal nanoparticles, including those nanoparticles prepared by precipitation.

When precipitation is used to prepare the particles, they may be prepared by precipitation from a solution that comprises at least a first surfactant, such as DTAB and related surfactants.

In the present invention, the pluralities of polymeric gel nanoparticles utilized in the formation of nanostructured polymeric networks may contain nanoparticles that are all substantially of the same particle sizes, or the particle diameters of the particles may be substantially different. Typically, preferred nanoparticles will have an average particle size of from about 1 to about 5000 nm, with average particle sizes of from about 5 to about 2000 nm, and those having average particle sizes of from about 10 to about 1000 nm being particular desirable. In certain embodiments, the plurality of polymeric gel nanoparticles will have particles of average sizes of from about 50 to about 500 nm in diameter.

### BRIEF DESCRIPTION OF THE DRAWINGS

The drawings form part of the present specification and are included to further demonstrate certain aspects of the present invention. The invention may be better understood by reference to one or more of these drawings in combination with the detailed description of specific embodiments presented herein.

FIG. 1A, FIG. 1B, and FIG. 1C. Structure of a polymer gel nanoparticle network. (FIG. 1A) Representative sketch of the gel nanoparticle network: The primary network (I) is crosslinked polymer chains in each individual nanoparticle, while the secondary network (II) is a system of crosslinked nanoparticles.

(FIG. 1C) Optical microscopic image of the HPC nanoparticle network in water at room temperature. The network was formed at 55° C. The white bar is 10 μm.

FIG. 2. Hydrodynamic radius distributions (f(Rₕ)) of HPC nanoparticles (C=8.94×10⁻⁶ g/ml) in deionized water at 25° C. These particles were prepared in 0.1 wt % HPC solution at various DTAB concentrations. CMC is the critical micelle concentration of DTAB in pure water at 25° C. and equals to 1.54×10⁻² mol/l.

FIG. 3. Hydrodynamic radius distributions (f(Rₕ)) of HPC nanoparticles (C=8.94×10⁻⁶ g/ml) in deionized water at 25° C. These particles were prepared using various HPC concentrations at 1 CMC of DTAB and at a reaction temperature 55° C.

FIG. 4. Hydrodynamic radius distributions (f(Rₕ)) of HPC nanoparticles (C=8.94×10⁻⁶ g/ml) in de-ionized water at 25° C. These particles were made at different reaction temperatures using a 0.1 wt % solution of HPC and 1 CMC of DTAB.

FIG. 5. The average hydrodynamic radius <Rₕ> of HPC nanoparticles changes as a function of crosslinking density and temperature in deionized water. The microgels with 10 wt % crosslinking density were prepared in a 0.5 wt % HPC solution using 1.5 CMC of DTAB and at a reaction temperature 65° C.

FIG. 6. The average hydrodynamic radius <Rₕ> of HPC nanoparticles changes as a function of temperature in de-ionized water and in 0.9 wt % NaCl aqueous solution, respectively.

FIG. 7A and FIG. 7B. The swelling and shrinking kinetics of a HPC nanoparticle network formed at room temperature. (FIG. 7A) Time-dependent swelling ratio of a sample that was cycled between two thermal baths set at 20° C. and 48° C., respectively (open circles with a solid line). The temperature profile is represented using a solid line. (FIG. 7B) Detailed plot of the shrinking kinetics of the sample. The sample had dimensions of 1 cm×1 cm×2.5 cm at room temperature in water. Vₒ represents the equilibrium volume of the sample at 20° C.

FIG. 8A and FIG. 8B. (FIG. 8A) Distributions of hydrodynamic radius of HPC nanoparticles prepared using methacrylated HPC. (FIG. 8B) Average hydrodynamic radius weighted by volume, scattering intensity and number of HPC nanoparticles prepared using methacrylated HPC vs. temperature.

FIG. 8C (Scheme 1). The HPC chain structure by attaching methacrylate moieties as side-groups allows for chemical crosslinking of the nanoparticles through a free radical polymerization process.

FIG. 8D (Scheme 2) Shows the general synthetic outline of a degradable crosslinker possessing the proper functionality for HPC modification.

FIG. 8E (Scheme 3) Illustrates the synthesis of modified HPC polymer with polymerizable groups that contain degradable, glycolate-type β-ester linkages.

FIG. 9A and FIG. 9B. (FIG. 9A) Degradation of degradable HPC nanoparticles at pH=9.1, 37° C. (FIG. 9B) Degradation of degradable poly-HPC nanoparticle at pH=1, 37° C.

FIG. 10A, FIG. 10B, and FIG. 10C. (FIG. 10A) Release of FITC labeled BSA from HCP nanoparticle networks with varying particle size at pH=7.4 and 37° C. (FIG. 1 OB). Initial rates or “burst” release of FITC labeled BSA from HPC nanoparticle networks with varying particle size at pH=7.4, 37° C. (FIG. 10C). Initial rates of release of FITC labeled BSA from HPC nanoparticle networks vs. particle size.

FIG. 11A, FIG. 11B, and FIG. 11C. (FIG. 11A) The release of BSA from within particles of a non-degradable HPC nanoparticle network (particle size=57 nm, total expected release=1875 ppm). (FIG. 11B) The release of BSA from within particles of a degradable HPC nanoparticle network (particle size=62 nm, total expected release=1875 ppm). (FIG. 11C) The release of BSA from between particles of a degradable HPC nanoparticle network (particle size=54 nm, total expected release=93 ppm).

FIG. 12. Release of BSA and BCG from HPC nanoparticle networks.

FIG. 13. Release and activity of HRP from HPC nanoparticle network.

FIG. 14A and FIG. 14B. Change in hydrodynamic radius of degradable poly-NIPA nanoparticle over time in phosphate buffered saline: (FIG. 14A) at pH=7.4 and 37° C. (FIG. 14B) pH=9 and 37° C.

FIG. 15. Release of bromocresol green from degradable poly-NIPA nanoparticles over time.

FIG. 16A, FIG. 16B and FIG. 16C. A NIPA-AA co-nanoparticle network exhibiting a blue color. (FIG. 16A) Z-average hydrodynamic radius distribution of NIPA-AA nanoparticles at 25° C. in water. The nanoparticles as basic blocks were then crosslinked to form a network. (FIG. 16B) At 22° C. the network swelled and exhibited a blue color; (FIG. 16C) at 37° C. it shrank and exhibited a white color. The brown bar represents 1 cm.

FIG. 17A and FIG. 17B. PVA-HPC nanoparticle networks (FIG. 17A) HPC-PVA nanoparticles, Synthesis and LLS characterization of PVA and HPC nanoparticles (FIG. 17B) HPC-PVA nanoparticle networks, Temperature dependence of PVA-HPC nanoparticle network.

### DESCRIPTION OF ILLUSTRATIVE EMBODIMENTS

Illustrative embodiments of the invention are described below. In the interest of clarity, not all features of an actual implementation are described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure.

The present invention relates to a class of materials based on the manufacture and covalently bonding of polymer gel nanoparticles together into networks. Some polymer gels that have been found to be useful in the present invention include hydroxypropyl cellulose (HPC), N-isopropylacrylamide (NIPA), and polyvinyl alcohol (PVA) and their derivatives.

In one embodiment, the present invention provides hydroxypropyl cellulose (HPC) nanoparticle compositions and methods for their synthesis utilizing precipitation. Although bulk HPC hydrogels, including homogeneous gels and porous gels, have been extensively studied and described in the prior art, the present invention provides the first synthesis of HPC nanoparticles. The manufacture of NIPA microgel particles starting with NIPA monomers has also been disclosed in the prior art.

By utilizing HPC polymer chains as a starting material instead of monomers, the present invention has demonstrated for the first time that HPC microgel particles can be readily produced. This was accomplished by dispersing HPC polymer chains in a surfactant solution and heating the mixture above the lower critical solution temperature (LCST) to yield colloidal particles that were subsequently crosslinked to form nanoparticles.

The present invention has also demonstrated for the first time that HPC polymer chains dispersed in a water-surfactant solution can collapse into colloidal particles at the LCST using a surfactant such as dodecyltrimethylammonium bromide (DTAB) in a concentration ranging from about 1 critical micelle concentration (CMC) to about 1.5 CMC. Below about 1 CMC, only very large particles (˜10 μm) were observed.

The collapsed polymer chains were stabilized by the charges on surfactant micelles that were attached to the polymer chains. After synthesizing HPC particles, the HPC nanoparticle dispersion was then dialyzed four times to remove surfactant and un-reacted chemicals. Then, the collapsed HPC polymer chains in each colloid were chemically crosslinked by divinylsulfone, forming nanoparticles.

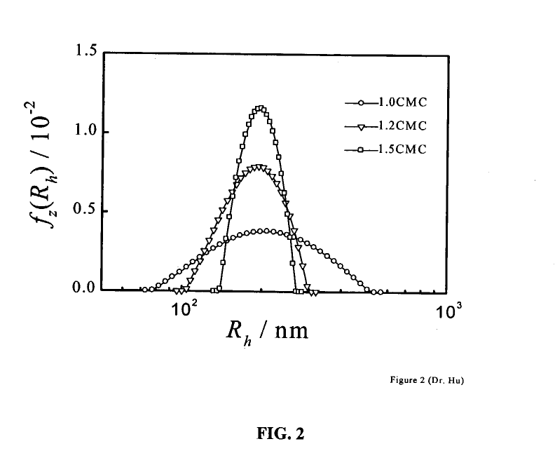

FIG. 2 shows hydrodynamic radius distribution (f(Rₕ)) of HPC microgels (C=8.94×10⁻⁶ g/ml) in deionized water at 25° C. These particles were prepared in 0.1 wt % HPC solution with various DTAB concentrations. In the surfactant concentration range studied, the average radii <Rₕ> of the nanoparticles were about 200 nm. However, the radius distribution f(Rₕ) becomes narrower with an increase of the surfactant concentration.

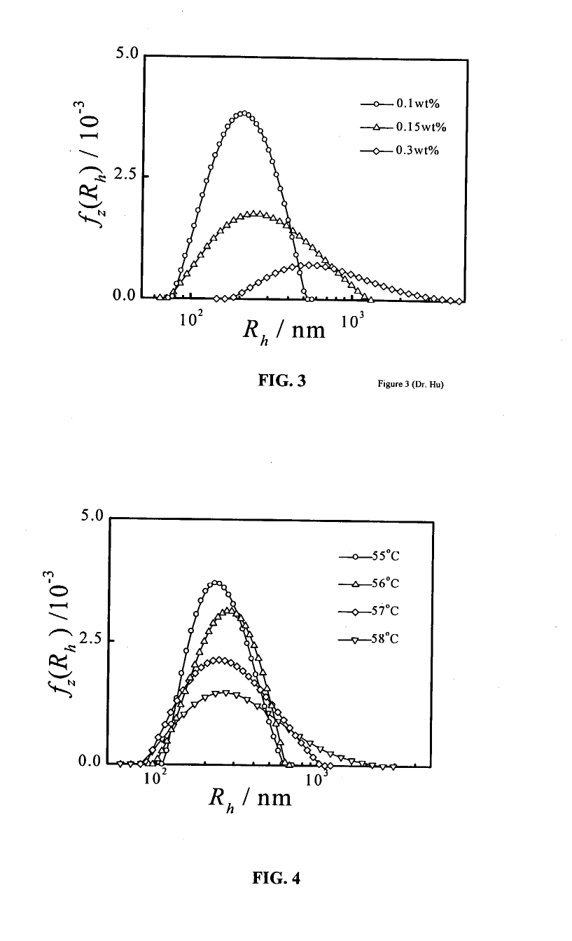

The size of HPC nanoparticles also depends on HPC polymer concentration. In this experiment, the HPC concentration varied from 0.1 wt % to 0.3 wt %, while the DTAB concentration and the reaction temperature were fixed at 1 CMC and 55° C., respectively. FIG. 3 shows hydrodynamic radius distributions (f(Rₕ)) of HPC nanoparticles (C=8.94×10⁻⁶ g/ml) in de-ionized water at 25° C. The average radius<Rₕ> of the microgel becomes larger and its distribution becomes broader with an increase in HPC concentration. This result might be explained in terms of the interaction between the DTAB surfactant and HPC. As the HPC concentration increases, the average number of absorbed surfactant aggregates on each HPC polymer chain should decrease, therefore reducing the inter-aggregate electrostatic repulsion force. This causes HPC linear chains to become more aggregated at a higher HPC concentration. Thus, the average radius <Rₕ> of the nanoparticle increases and its distribution becomes broader.

The reaction temperature plays an important role in the formation of the HPC nanoparticles. FIG. 4 shows hydrodynamic radius distributions (f(Rₕ)) of HPC nanoparticles (C=8.94×10⁻⁶ g/ml) in de-ionized water at 25° C. These particles were made at various reaction temperatures in 0.1 wt % HPC solution and at a CMC concentration of DTAB. The reaction temperature at which microgels form is in a small range within about three degrees above the LCST, which is 55° C. for this dispersion. Below the LCST, we did not observe formation of HPC nanoparticles. In this range studied, as the reaction temperature increases, the average radius of the resultant nanoparticles becomes larger and the radius distribution becomes broader.

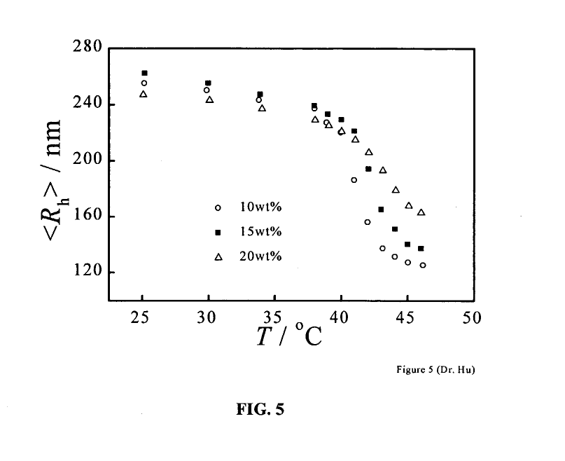

The average hydrodynamic radius may be plotted as a function of temperature as shown in FIG. 5. Although up to 20 wt % of crosslinker relative to the HPC is used during synthesis, the inherent swelling and solubility properties of the non-crosslinked linear HPC polymer are expected to dominate with respect to gel swelling. The average molar mass of the segment between two neighboring crosslinking points, (\overline{M}_{c},) is inversely proportional to the crosslinking concentration. As a result, the degree of swelling at room temperature and the size change below and above T_{c} decrease as the crosslinking concentration increases.

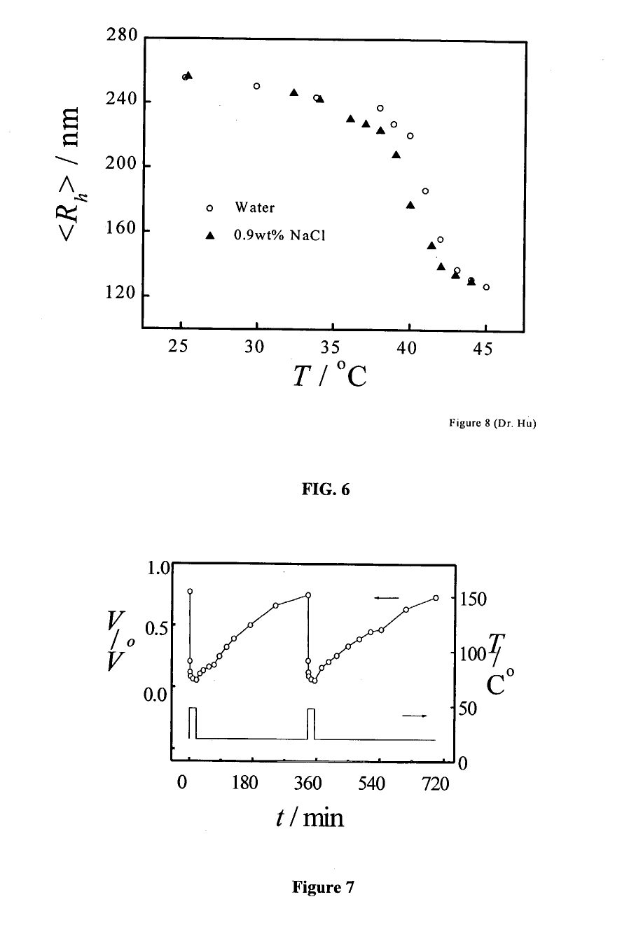

The salt effect on the phase transition temperature of HPC nanoparticles in water was also investigated. FIG. 6 shows the average hydrodynamic radius <Rₕ> as a function of temperature for HPC nanoparticles (C=8.94×10⁻⁶ g/ml) in water and in 0.9 wt % NaCl solution (0.15 mol.l⁻¹, physiological ionic strength), respectively. T_{c} is about 41° C. for the nanoparticles in pure water, while it is about 39° C. for the nanoparticles in 0.9 wt % NaCl. The decrease of T_{c} with the addition of NaCl may be a result of inorganic ions forming hydrates through ion-dipole interactions. The disturbance of water structure by adding NaCl in HPC dispersion induces contact between HPC polymer chains, causing a decrease of T_{c} of HPC nanoparticles. Combining the temperature-responsive volume change, the biocompatibility and low toxicity of HPC, and the uniform and small particle size, the resultant HPC nanoparticles could be particularly useful as materials for the controlled delivery of drugs or other active compounds.

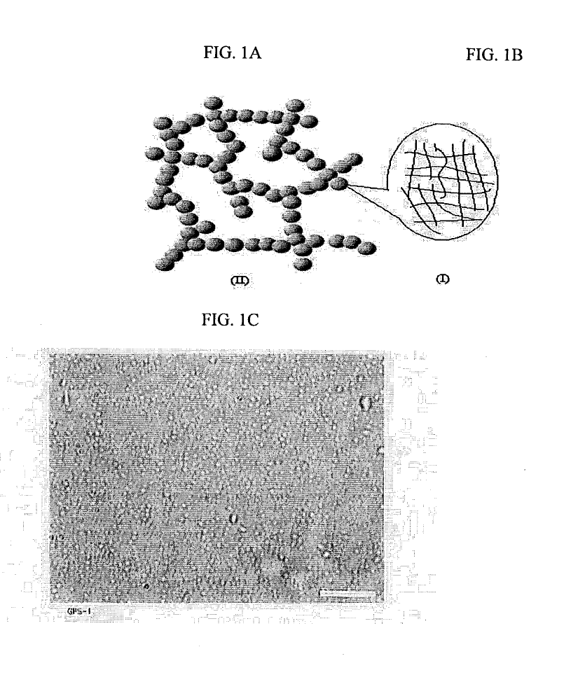

The average hydrodynamic radius (<Rₕ>) and Rₕ distribution function, f(Rₕ) of these nanoparticles was characterized using an ALV laser light scattering system. <Rₕ> ranged from 120 nm to 250 nm depending on chemical composition and reaction temperature and conditions. The residual hydroxyl groups on the surfaces of neighboring HPC nanoparticles were then bonded together to form a network. In contrast to other well-known colloidal aggregates, these nanoparticles cannot be re-dispersed into solution. The optical microscopic image of a HPC nanoparticle network in water at room temperature is presented in FIG. 1C.

In another embodiment of the present invention, the resulting HPC gel nanoparticle network exhibits new swelling kinetics. As shown in FIG. 7A and FIG. 7B, the swelling ratio of a HPC nanoparticle network with dimensions of 1 cm×1 cm×2.5 cm was measured as a function of time after the sample was cycled between two thermal baths set at 20° C. and 48° C. This sample was synthesized using the same method as described above except that crosslinking between the gel nanoparticles was performed at room temperature. The HPC nanoparticle network swelled at 20° C., but collapsed very quickly at 48° C., which was above HPC volume phase transition temperature T_{c} of 41° C. as reported in the literature. The HPC nanoparticle network exhibited a distinctive asymmetric kinetics: its shrinking rate was faster by about two orders of magnitude than the shrinking rate of a conventional homogeneous gel of similar chemical composition and dimensions. However, its swelling rate was not significantly higher.

The fast shrinking rate arises from the unique structure of the nanoparticle network. It is well known as stated in related scientific publications that the shrinking or swelling time of a gel is dependent on the square of the smallest linear dimension and is very slow for a bulk gel. The nanoparticles in the network are so small that they should very quickly respond to an external stimulus. Therefore, the shrinking and swelling kinetics are mainly controlled by movement of water through the spaces between nanoparticles. Such spaces may be better connected in the shrinking process than in the swelling process, resulting in the faster responsive shrinking rate. These nanoparticle networks provide advantages with respect to a highly uniform and easily tunable mesh size when compared to other fast responsive gels reported in the literature that were produced by either creating pores in a gel or grafting hydrophobic chains into the gel. For example, the pore size in a nanoparticle network can be easily and well controlled by varying either nanoparticle size or the average number of nearest neighbors.

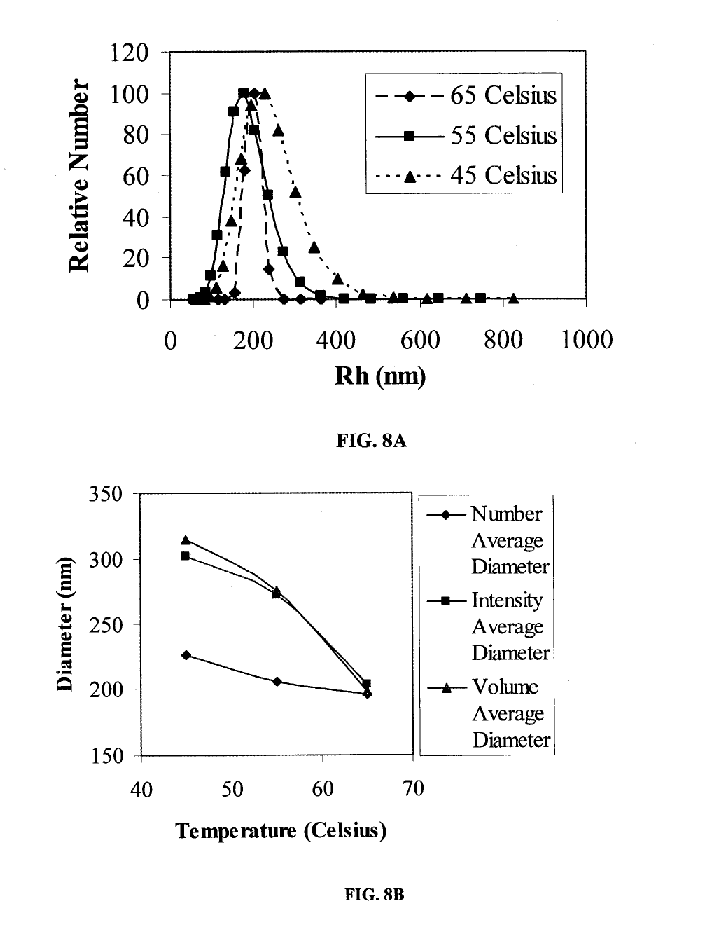

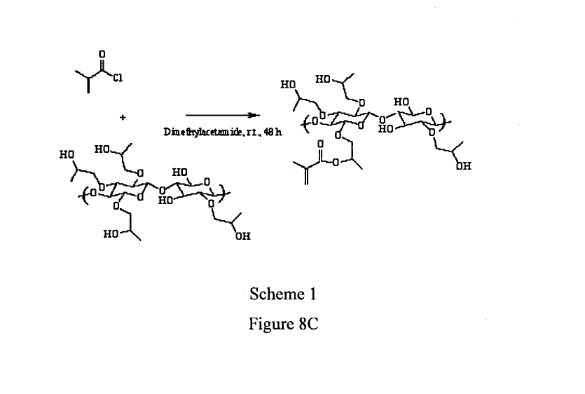

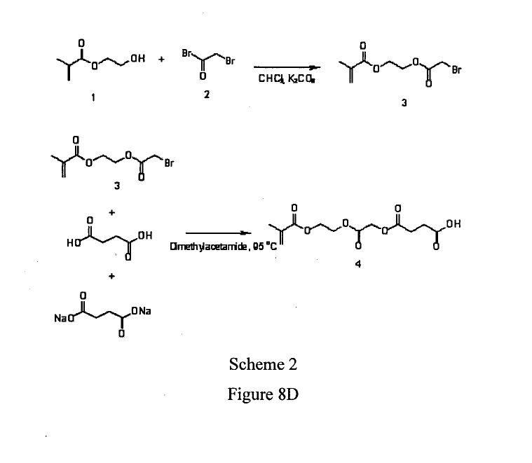

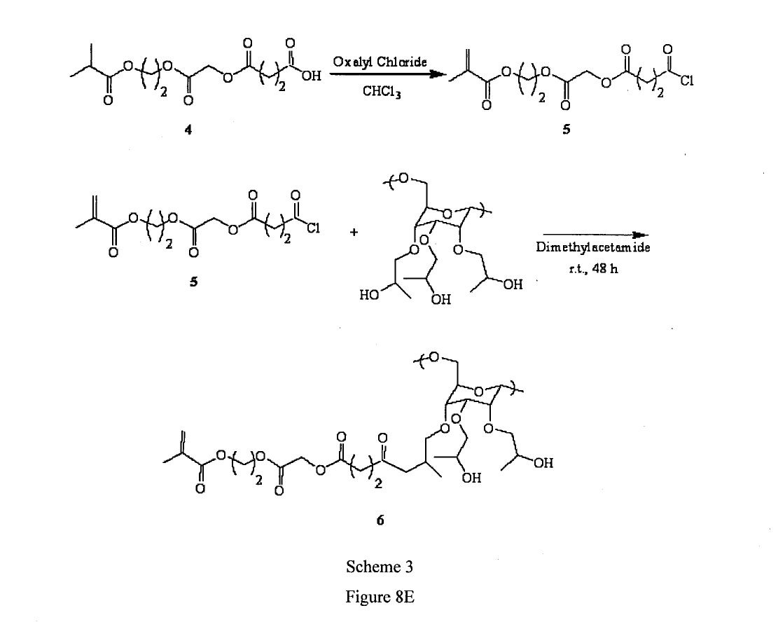

A further embodiment of the invention is the preparation of HPC nanoparticles using a surfactant-free method. Modifying the HPC chain structure by attaching methacrylate moieties as side-groups allows for chemical crosslinking of the nanoparticles through a free radical polymerization process. Scheme 1 (See FIG. 8C) shows the general synthetic outline for this modification.

In this particular case, the methacrylate groups provide non-degradable crosslinking of HPC nanoparticles. An aqueous solution of the modified HPC of Scheme 1 is prepared without surfactant. As the solution temperature is raised above the LCST, individual HPC chains aggregate into nanoparticles. Addition of potassium persulfate initiates radical polymerization of methacrylate side-groups of the modified HPC resulting in nonreversible nanoparticle formation. The formed nanoparticles are easily collected by ultracentrifugation.

FIG. 8A shows plots of three distributions of nanoparticle sizes from three different nanoparticle populations. These three samples were prepared at three different temperatures. The data clearly indicate that lower temperatures lead to broader distributions of nanoparticle size and also larger average particle sizes. Therefore, simply raising or lowering the temperature allows for tailoring of HPC nanoparticle sizes when using this strategy. FIG. 8B shows three plots of the average nanoparticle size vs. temperature. The three different plots correspond to three different weighting methods used to determine the average: numbered average, volume average and intensity average. Note the convergence of the plots at higher temperatures. This indicates a narrowing of the distribution in particle sizes at higher temperatures.

Another embodiment of the invention is the preparation of degradable nanoparticles using a degradable crosslinker. Scheme 2 (See FIG. 8D) shows the general synthetic outline of a degradable crosslinker possessing the proper functionality for HPC modification. This crosslinker is an asymmetric derivative of crosslinkers disclosed earlier (U.S. patent application Ser. No. 09/338,404, specifically incorporated herein by reference in its entirety without disclaimer). The hydrolytic susceptibility of the β-ester is far greater than those of normal esters at physiological pH. Hence, their utility in controlled release applications of various pharmaceuticals is expected.

Scheme 3 (See FIG. 8E) illustrates the synthesis of modified HPC polymer with polymerizable groups that contain degradable, glycolate-type β-ester linkages. HPC modified in this way can also be used to prepare nanoparticles without the need for surfactant. The methods are identical to those used to prepare non-degradable HPC nanoparticles, and the nanoparticles also show similar trends between nanoparticle size and temperature of synthesis.

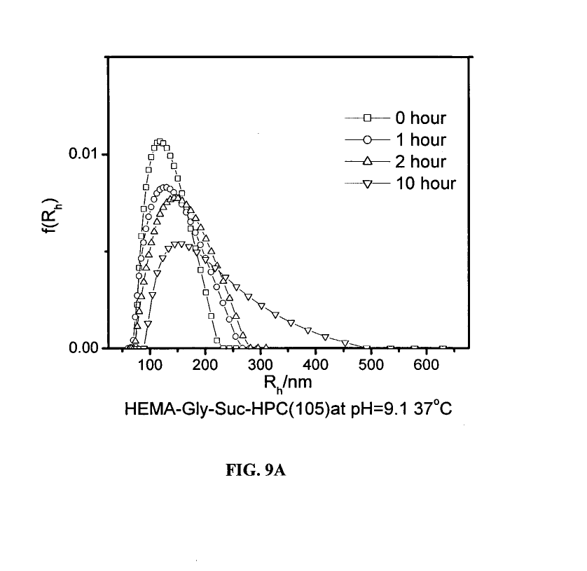

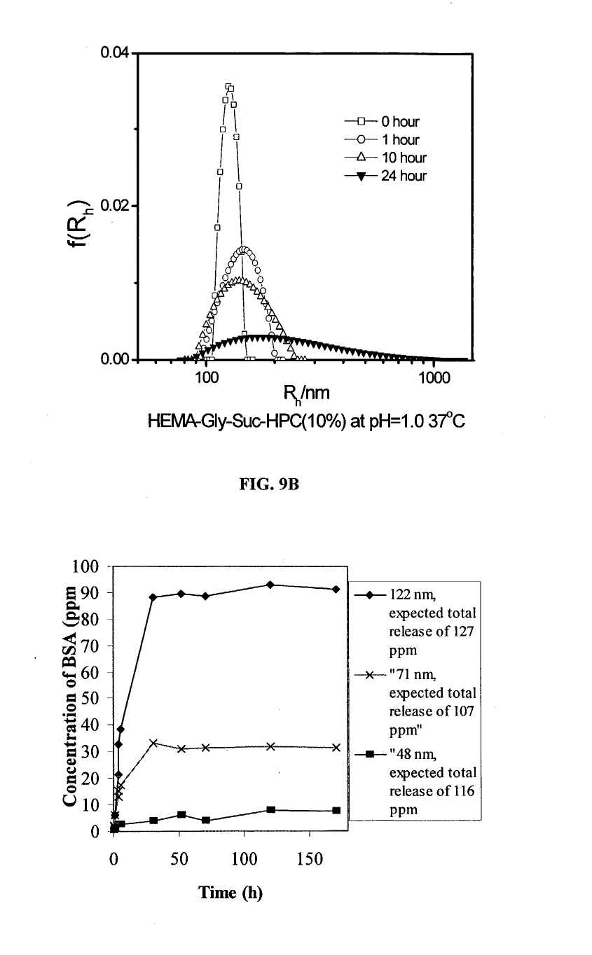

The degradable characteristics of these nanoparticles are illustrated in FIG. 9A and FIG. 9B. Both sets of data are from pH's that accelerate the degradation of the nanoparticles. In both cases there is a general broadening of the particle size distributions. Since swelling capacity of bulk polymers is dependent on crosslinking density (i.e., as crosslinking density decreases swelling capacity increases), this broadening is expected. As the number of crosslinks decreases due to degradation within the nanoparticle, the swelling capacity of the nanoparticle increases. Furthermore, it is envisioned as the number of crosslinks decreases over time, the diffusion of entities from within the particles will be greater. This should have valuable impact on the application of controlled delivery for the device.

Another embodiment of the invention is the preparation of nanoparticle networks from nanoparticles prepared from the surfactant-free method. Residual methacrylate groups are present both within and on the exterior of HPC nanoparticles. These residual methacrylate groups are used to covalently link the HPC nanoparticles together into a network. As the nanoparticles are collected by ultracentrifugation, potassium persulfate in addition to sodium metabisulfite are added. Hence, the residual methacrylate groups on the exterior of the nanoparticles are linked together to form the secondary structure of the network.

In another embodiment of the present invention, the polymer gel nanoparticle network is used as vehicles for the controlled-, time- and/or sustained-release of compositions comprised within the system of nanoparticles. For example, chemical entities, such as compounds, pharmaceuticals, drugs, and such like can be entrapped between particles i.e., in the secondary network that is a crosslinked system of the nanoparticles. The mesh size of the secondary network depends on size of the nanoparticles. The interstitial space (mesh size) decreases as the particle size decreases for closely packed polymer nanoparticle networks. This property can be used to control or regulate the release rate of chemical entities from the network.

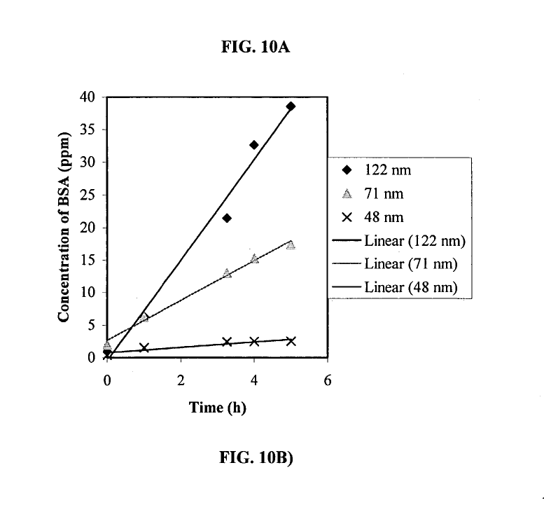

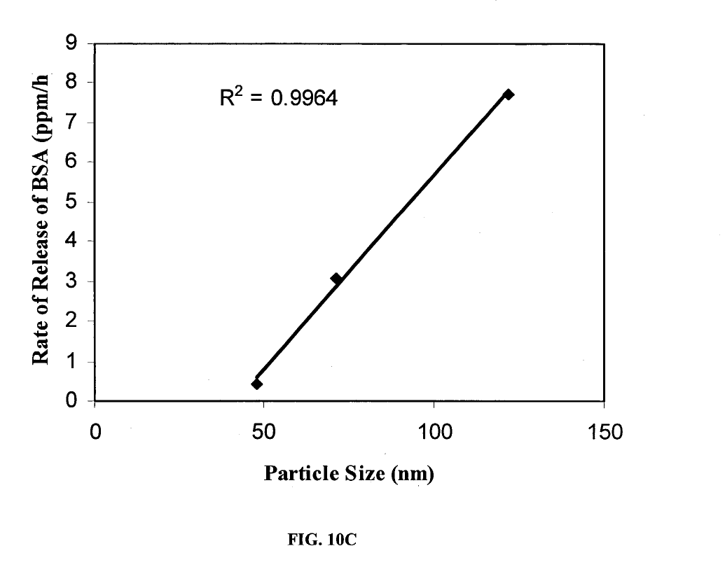

In an illustrative example, FIG. 10A shows the ability of the compositions of the invention to release an entrapped compound over a period of time. In FIG. 10A, plots for the release of a labeled protein, in this case fluorescein-labled (FITC) bovine serum albumin (BSA), from HPC nanoparticle networks of different particle size compositions. All three samples show a “burst” release in the first five hours of release. However, for the network with the smallest size particles (approximately 48 nm) the “burst” was significantly lessened. Although, the complete release of BSA was not seen in any of the samples, the amount of BSA released from the nanoparticle network was clearly dependent upon the sizes of the hydrogel nanoparticles contained within the network.

FIG. 10B shows the “burst” release profiles of each of the samples in FIG. 10A. These data suggest a relationship exists between the selected nanoparticle size and the initial rate of release of the entrapped protein, (e.g., BSA). FIG. 10C is a plot of the slopes of each of the plots in FIG. 10B as a function of particle size. These data demonstrate that the diffusion rate of the entrapped protein (in this case, BSA) within the nanoparticle network can be controlled by controlling the network mesh size and by manipulating the size of the nanoparticles used to form the network.

The present invention also demonstrates that chemical entities can also be entrapped within the primary structure of the particles that comprise the network, and can thus be readily incorporated into a nanoparticle network by utilizing nanoparticles that contain the selected chemical entities in the formation of the network. Since the chemical entities of interest may be initially located within the particles themselves, diffusion of the chemical(s) from the particles is primarily effected by the crosslinking density or the rate of degradation of the network due to the presence and/or quantity of degradable crosslinks within the nanoparticle matrix. Once the chemical entity has exited the plurality of nanoparticles that comprise the network, its rate of release from the network will be governed primarily by the secondary structure of the overall matrix (i.e., the mesh and/or particle sizes comprising the matrix).

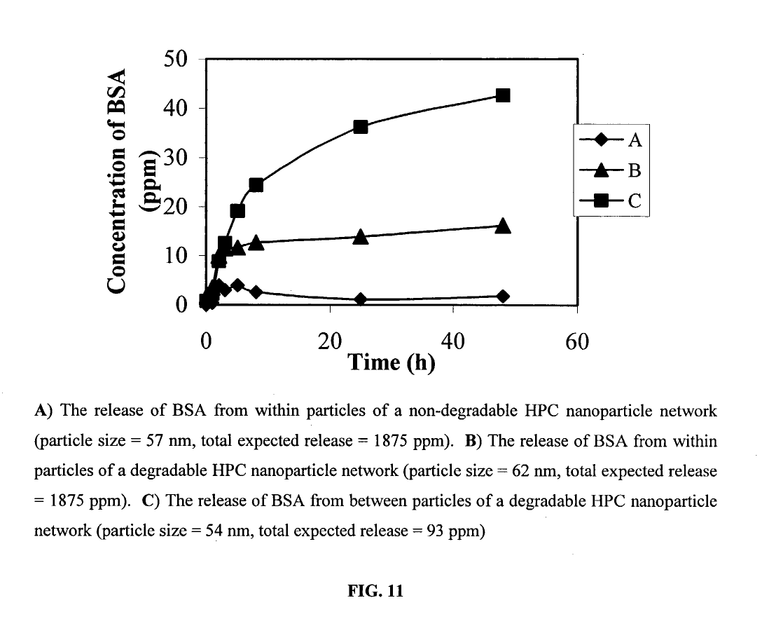

FIG. 11A, FIG. 11B, and FIG. 11C contain plots demonstrating the release of a selected protein (in this case, BSA) from three different matrices: FIG. 11A shows the results of substrate release when the BSA is contained within the particles in a matrix without degradable links. FIG. 11B shows the results of substrate release when the BSA is contained within the particles in a matrix that contains degradable crosslinks. FIG. 11C shows the results of substrate release when the BSA in entrapped between the particles that comprise a degradable crosslinked matrix. Clearly apparent is the affect of BSA residing within the particle as opposed to between the particles. The release of BSA from within particles of degradable HPC nanoparticle networks had an almost completely negligible “burst” release when compared to the total amount of BSA loaded into the network. Furthermore, the release appears to be continuing, probably as a result of the slow degradation of the nanoparticles and subsequent release of BSA. This is compared to the non-degradable HPC nanoparticle network where the release has completely stopped.

Contrasting this is the significant “burst” release of BSA from degradable HPC nanoparticle networks where the BSA originates from the secondary structure (i.e., between particles). The fact that the network is degradable has little to no effect on the release rate of BSA. This shows that the diffusion of chemical entities within the secondary structure is not affected by the degradation of the network, since the diffusion is much faster than degradation. This however would not be the case where smaller particle sizes, hence smaller mesh size would occlude smaller chemical entities. It should be noted that only about 50% of the BSA has been released from the secondary structure of this network by diffusion. The remaining release will most likely be affected by the degradation of the network.

In the present invention, the polymer gel nanoparticle network has two levels of structural difference; the primary network and the secondary network. The mesh size of the primary network is much smaller than that of the secondary network. This structural property leads to a device that can be used for simultaneous release a small and large molecules with distinct release profiles. As a demonstration, a HPC nanoparticle network was formed with a small molecule (the dye, bromocresol green [BCG]) entrapped into the particles and large chemical entity (a protein, BSA) entrapped between the particles. The synthetic procedure is detailed in Example 11.

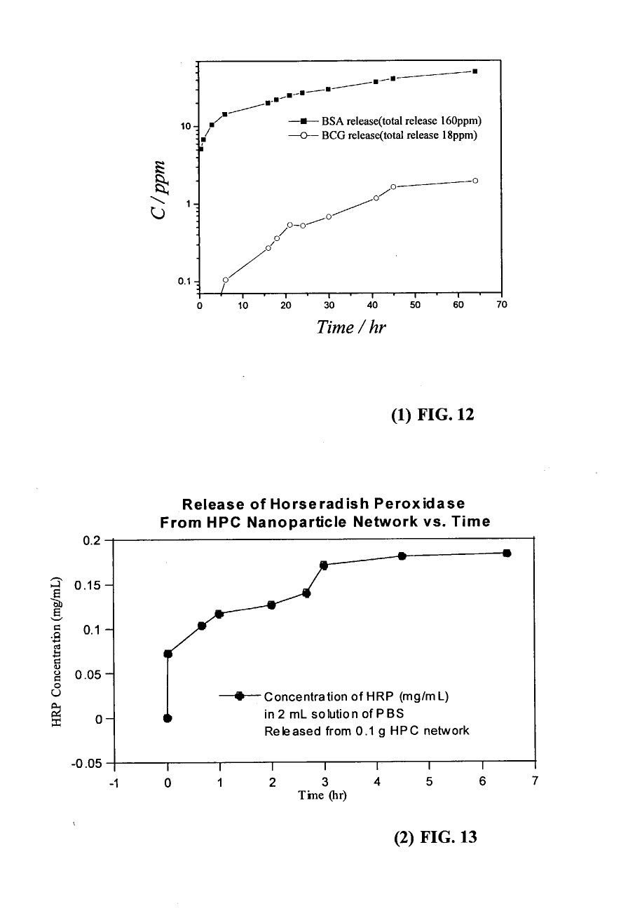

This nanoparticle network was then immersed in a PBS buffer solution at pH=7.4 and at 37° C. The time-dependent release of BCG and BSA was monitored by a UV-Vis spectroscopy. The characteristic absorptions for the BSA and the BCG were demonstrated at 496 nm and 620 nm, respectively. As shown in FIG. 12, the HPC nanoparticle network could release both a relatively small molecule (BCG) and a relatively large molecule (BSA) substantially simultaneously. These data show a sharp departure from normal diffusion kinetics seen normally with bulk gels. BSA's release from the network is almost an order of magnitude greater than that of BCG, though the size of BCG is over two orders of magnitude smaller. This shows that contained within the same network are two distinct release sites operating under two distinct mechanisms, further illustrating the potential versatility of the invention in the arena of controlled delivery, particularly when the controlled release of two or more chemical entities from within a single nanoparticle network matrix is contemplated.

The controlled release of an active compound can require that some molecules, such as proteins, be protected from proteolytic enzymes in vivo. As a demonstration of the ability of a network to load an active drug and protect the activity of that drug, the enzyme horseradish peroxidase was loaded into one of the disclosed gel networks of the invention, and then subsequently exposed to the protease trypsin. A control gel immersed in phosphate buffered saline (pH=7.4) showed that the enzyme activity remained at 98% of the free enzyme with release from the network. Next HPC nanoparticle networks containing HRP were immersed in a solution containing trypsin for 5, and 30 minutes and then removed from the trypsin solution, rinsed and immersed in phosphate buffered saline (pH=7.4). The data in Example 12 shows the free enzyme activity in the presence of trypsin after 5 and 30 minutes, and the activity of HRP released from the network in PBS after exposure of the network to trypsin. This data clearly shows that although the activity of the enzyme is compromised in the presence of the protease, the activity of the HRP remaining in the network remains at 95% or more with time. Details of this experiment are found in Example 12.

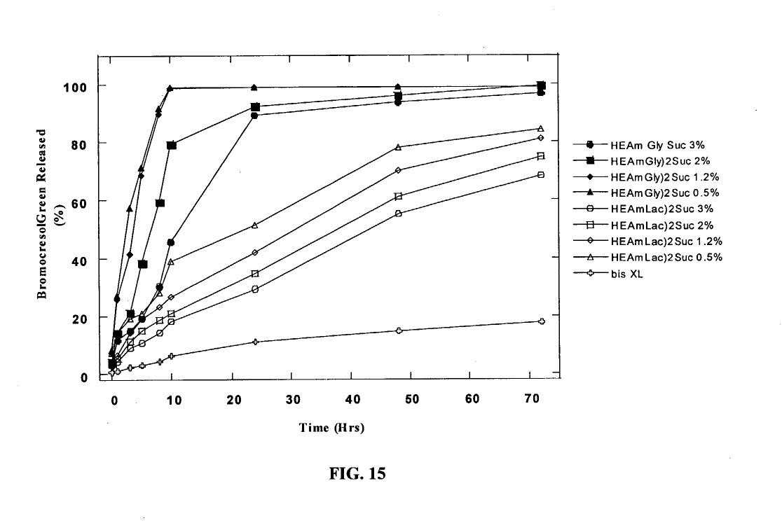

Temperature responsive degradable hydrogel nanoparticles can be used to control the release of a small molecule trapped within the hydrogel body. As a demonstration, the dye molecule bromocresol green was trapped into N-isopropylacrylamide hydrogels crosslinked with degradable and non-degradable crosslinkers. The exact synthetic route is detailed in Example 14. As shown in FIG. 15, the release of the small molecule can be controlled by the nature and mole percentage of crosslinker in the hydrogel nanoparticle.

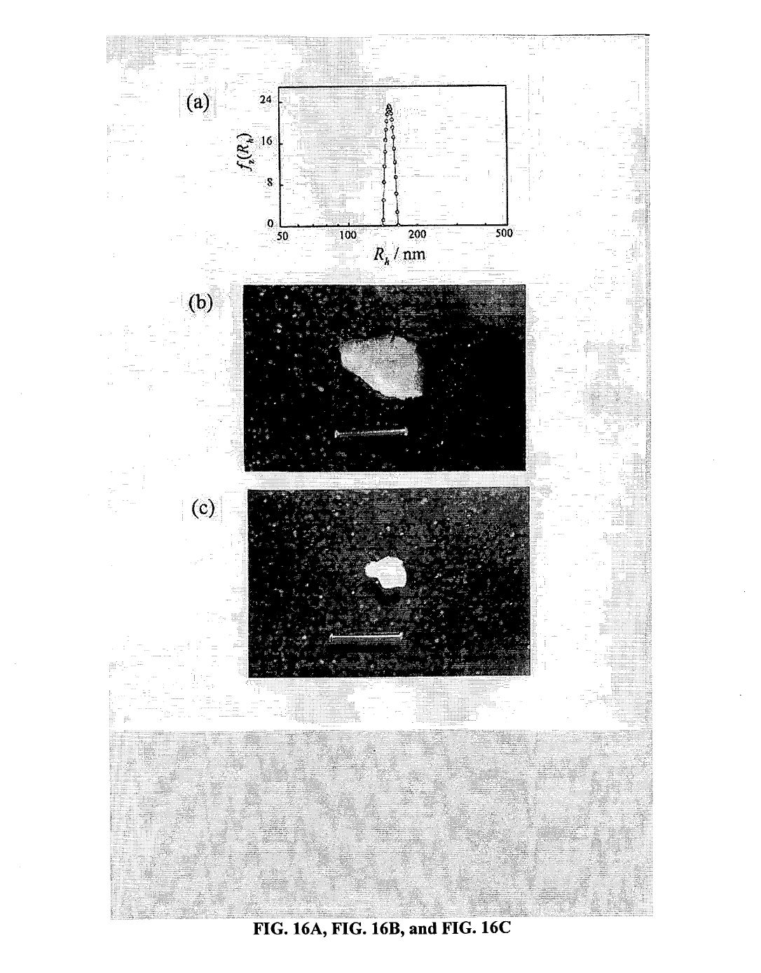

The nanoparticle networks disclosed in the present invention can be made to retain some inherent properties that the bulk polymeric dispersions exhibit. As a demonstration, nanoparticles of co-polymer N-isopropylacrylamide (NIPA, molar fraction of 96%) and acrylic acid (AA, 4%) were produced using an emulsion method. The exact synthetic route is detailed in Example 15. The NIPA has a thermally responsive property, while the AA provides carboxyl groups (—COOH) suitable for subsequent crosslinking sites. The resulting poly NIPA/AA nanoparticle had an average radius of 153 nm at 25° C. in water as shown in FIG. 16A. Upon extensive ultra-centrifugation, a concentrated NIPA/AA nanoparticle dispersion was obtained and it exhibited a bright blue color. Epichlorohydrin was then added and the dispersion heated at 98° C. for 10 hours. The resulting nanoparticle network was then purified using acetone and water.

This NIPA/AA nanoparticle network not only retained the blue color of the dispersion, but also had excellent mechanical stability that is not found with conventional dispersions. As shown in FIG. 16B, the nanoparticle network kept its shape in water without external support of a container at room temperature. In contrast to conventional gels that are colorless, this nanoparticle network exhibited a blue color. When the temperature was increased to 37° C. (FIG. 16C), that is, above the volume phase transition temperature (T_{c}=34° C.) of the NIPA gel, the network completely shrank and exhibited a white color due to non-selective light scattering by microdomains formed during the volume transition. Combining the environmentally responsive color and volume changes, the nanoparticle networks disclosed in the present invention may potentially function as a display element or as a sensor for biological and/or medical applications.

In addition, hydrogels are typically clear without the addition of an external coloring agent after they fully swell in water. The polymer gel nanoparticle networks described in the present invention exhibit a distinguishable and unique color. This color can enhance contrast so that the gel can be easily identified when it is immersed in water or other solvent. Furthermore, the distinct color and color uniformity can be used as a quality control or analytical tool to characterize and describe a specific gel structure. Color uniformity is indicative of a homogeneous gel structure and provides a way to assess the reproducibility and viability of the manufacturing processes used to produce such gels.

It is also expected that such nanoparticle networks could be used to stabilize crystal colloid arrays. Conventional colloidal crystal arrays as disclosed in the cited prior art have found little practical application due to their poor mechanical and thermal stability. To overcome this shortcoming, such arrays can be embedded into a gel matrix and this process has been discussed in another reference paper. Using the teachings disclosed in the present invention, these colloidal crystal arrays could also be stabilized by directly linking nanoparticles through chemical bonds without introducing another gel matrix.

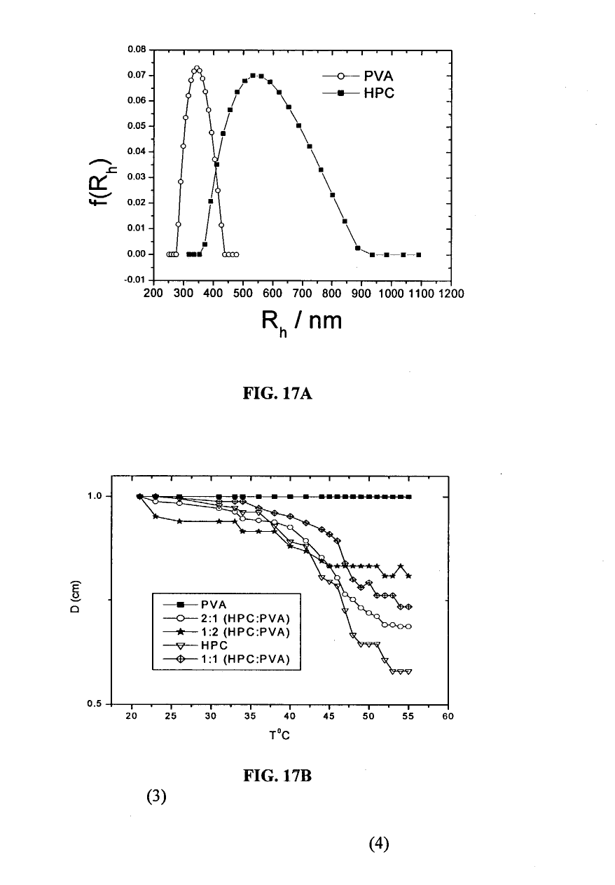

The form or type of a polymer gel nanoparticle network will depend to a large extent on the use for which it is intended. One suitable form is a gel comprising two different gel nanoparticles. Different nanoparticles composed from either monomers or polymers that have inherent different physical properties can be used as basic building blocks for the synthesis of co-nanoparticle networks. This idea was first demonstrated by synthesizing nanoparticles of poly(vinyl alcohol) (PVA) and HPC, as shown in FIG. 17A and then covalently bonding them together. The synthesis of HPC nanoparticles was mentioned previously and shown in Example 1. The PVA nanoparticles were prepared using a surfactant-free method, then crosslinked, and the complete synthetic scheme is detailed in Example 16. The reaction lasted about four hours and the resulting nanoparticle dispersion was dialyzed five times. The PVA and HPC nanoparticles were then mixed. Crosslinking was performed by adding divinylsulfone to the PVA-HPC dispersion at pH=12 at 45° C. according to the following mechanism:

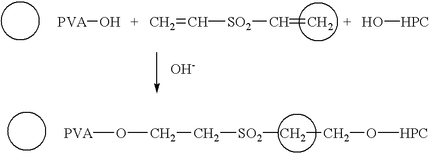

The PVA/HPC co-nanoparticle network formed within 1 hour. In contrast to well-established co-polymerization of different monomers to produce block copolymers, the co-nanoparticle network retains some inherent, physical properties native to the individual, non-combined nanoparticles. As a result, such a network could perform multifunctional tasks. Using this PVA/HPC co-nanoparticle network as an example, the PVA nanoparticles could act as a bioadhesive agent due to its inherent mucoadhesive property while the HPC nanoparticles could serve as a drug carrier to provide temperature-controlled drug release as result of its temperature responsive attribute.

In this invention, temperature dependent swelling ratios of the HPC-PVA co-nanoparticle networks are measured and shown in FIG. 17B. It can be seen from the figure that the HPC nanoparticle network has the lowest LCST, while PVA exhibit no temperature responsive property. As PVA nanoparticle concentration increases, the phase transition temperature increases.

In addition, the bioadhesion of the PVA could be further enhanced due to the increased surface area resulting from the PVA nanoparticle structures. Furthermore, as temperature increases, the PVA nanoparticles will expand while the HPC nanoparticles will shrink, resulting in a temperature-tunable heterogeneity on a nanometer scale. This type of co-nanoparticle network technology may provide many new nanostructured polymeric materials for use in a wide range of diversified commercial applications.

Another potential drug delivery application can be envisioned within the scope of this invention. Nanoparticles of N-isopropylacrylamide or another temperature responsive polymer can be covalently crosslinked together, with, for example, nanoparticles produced from PVA. This network can be exposed to a solution containing a large quantity of pharmaceutical compound or other active material. The solution can be water or an organic solvent, as long as the solvent does not have any deleterious effects on the active substance. The only other requirement is that the co-nanoparticle network must swell upon exposure to the solvent. At room temperature, the drug or other active compound will be drawn into the nanoparticle network by diffusion. Then, the temperature is increased above the LCST (˜34° C.) and the N-isopropylacrylamide nanoparticles will shrink and potentially prevent the drug from leaving the co-nanoparticle network. If successful, this process can be repeated several times to maximize drug loading and to also purify the co-nanoparticle network. Upon injection or infusion into the body, the temperature responsive nanoparticles will shrink again, leaving holes in the network for the drug to escape. It can also be envisioned that a biodegradable crosslinker be used in conjunction with this co-nanoparticle network to alter and control the biodegradable properties of the network and therefore the release rate of the active compound. In addition, it is apparent that the actual size of the nanoparticles used to create a co-nanoparticle network and the type of packing arrangement created during crosslinking will also affect the drug release rate.

Following the teachings of this invention, nanoparticle networks of various compositions and/or different biodegradable crosslinker types and amounts containing a specific active compound or a mixture of actives may also be combined together. The resulting combination may provide an overall synergistic effect of drug delivery due to the differences in biodegradability rates for each network. It can be envisioned that the unwanted “burst effect” common with drugs entrapped in erodible matrix devices can be easily eliminated or minimized using a mixture of nanoparticle networks described in the present invention.

One can also foresee that these unique nanoparticle networks may be designed with properties suitable for use as an environmental cleanup material. For example, a nanoparticle network can be fabricated with free ionic charges available to complex with metal contaminants present in water. The extraction of these contaminants would be very efficient due to the large surface area of these networks and would probably be most effective in the cleanup of radioactive waste water. These networks can also be designed to degrade if desired at a specific pH or other external environmental condition to release and concentrate the toxic contaminants in a defined area.

Therapeutic and Diagnostic Kits

In another embodiment, the invention provides therapeutic kits and medicaments that comprise at least one or more of the disclosed nanoparticles, crosslinked nanoparticle compounds, nanoparticle matrices, networks, or a combination thereof, in combination with instructions for using the compositions in the administration of one or more pharmaceuticals or medicaments in the diagnosis, treatment, prophylaxis, or amelioration of symptoms from one or more mammalian diseases, dysfunctions or disorders. Alternatively, the invention provides kits that comprise at least one or more of the disclosed drug delivery compositions in combination with instructions for using the compositions in the preparation of a pharmaceutical composition for use in therapy. Likewise, the invention provides kits that comprise at least one or more of the disclosed compositions in combination with instructions for using the manufacture of a medicament for the therapy of animals, and in particular, human and/or non-human mammals.

The invention also encompasses one or more of the disclosed nanoparticle matrix compositions together with one or more pharmaceutically-acceptable excipients, carriers, diluents, adjuvants, and/or other components, as may be employed in the formulation of particular drug delivery formulations, and in the preparation of therapeutic agents for administration to a mammal, and in particularly, to a human, for the treatment, diagnosis, prophylaxis, or amelioration of one or more diseases, dysfunctions, or disorders. In particular, such kits may comprise one or more of the disclosed nanoparticle matrix compositions in combination with instructions for using the compositions in the administration to humans, or animals under veterinary care, one or more pharmaceutical formulations of the disclosed compositions, and may typically further include containers prepared for convenient commercial packaging.

As such, preferred animals for administration of the pharmaceutical compositions disclosed herein include mammals, and particularly humans. Other preferred animals include murines, bovines, equines, porcines, canines, and felines. The composition may include partially or significantly purified nanoparticle network compositions that comprise one or more therapeutics or medicaments, either alone, or in combination with one or more additional active ingredients, which may be obtained from natural or recombinant sources, or which may be obtainable naturally or either chemically synthesized, or alternatively produced in vitro from recombinant host cells expressing DNA segments encoding such additional active ingredients.

Therapeutic kits may also be prepared that comprise at least one of the compositions disclosed herein and instructions for using the composition as a therapeutic agent. The container means for such kits may typically comprise at least one vial, test tube, flask, bottle, syringe or other container means, into which the disclosed composition(s) may be placed, and preferably suitably aliquoted. Where a second composition is also provided, the kit may also contain a second distinct container means into which this second composition may be placed. Alternatively, the plurality of biologically active compositions may be prepared in a single pharmaceutical composition, and may be packaged in a single container means, such as a vial, flask, syringe, bottle, or other suitable single container means. The kits of the present invention will also typically include a means for containing the vial(s) in close confinement for commercial sale, such as, e.g., injection or blow-molded plastic containers into which the desired vial(s) are retained.

Pharmaceutical Compositions

In certain embodiments, the present invention concerns formulation of one or more compositions comprising at least a first nanoparticle, conjugated nanoparticle, crosslinked nanoparticle, or a nanoparticle network disclosed herein in the manufacture of medicaments, and the preparation of pharmaceutically acceptable solutions for use in therapy of humans and non-humans animals, and administration of one or more of such compositions to a cell or an animal, either alone, or in combination with one or more other modalities of therapy, treatment, diagnosis, or amelioration of symptoms.

It will also be understood that, if desired, the disclosed nanoparticulate compositions described herein may be used to deliver one or more biologically-active, or therapetuically-effective agents, either alone or in combination with one or more other therapeuticums, as well, such as, e.g., proteins or polypeptides or various pharmaceutically-active agents. In fact, there is virtually no limit to the compounds or compositions that may be formulated with the disclosed nanoparticle compositions, such that other components that may also be included, given that the additional agents do not cause a significant adverse effect upon contact with the target cells or host tissues. The disclosed compositions may thus be used in the delivery of compounds, therapeutics, either alone, or along with various other agents as required in the particular instance that may be contemplated by one of skill in the art having the benefit of the present teachings. Such compounds or compositions may be commercially obtained, synthesized, and/or purified from host cells or other biological sources, or alternatively may be chemically synthesized as described herein. Likewise, such compositions may comprise pharmaceuticals, compounds, and such like.

Formulation of pharmaceutically-acceptable excipients and carrier solutions is well-known to those of skill in the art, as is the development of suitable dosing and treatment regimens for using the particular compositions described herein in a variety of treatment regimens, including e.g., oral, parenteral, intravenous, intranasal, and intramuscular administration and formulation. Such formulations may be used to prepare the disclosed nanoparticle networks in the necessary buffers, diluents, physiologically-acceptable carriers, etc. that may be required when the disclosed compositions are contemplated for administration to an animal or in particular, a human.

Typically, the disclosed nanoparticle matrix and structured nanoparticle networks disclosed herein, when used in drug delivery, and/or controlled-release regimens, may be formulated such that the networks and nanoparticle formulations will contain at least about 0.1% of the active compound entrapped or contained within the particles or the particle matrix, although the percentage of the active ingredient(s) may, of course, be varied and may conveniently be between about 0.5% or 2% and up to and including about 70% or 80% or more of the weight or volume of the total nanoparticulate matrix formulation. Naturally, the amount of active compound(s) in each therapeutically useful nanoparticule composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In certain circumstances it will be desirable to deliver the pharmaceutical compositions disclosed herein parenterally, intravenously, intramuscularly, or even intraperitoneally as described in U.S. Pat. Nos. 5,543,158; 5,641,515 and 5,399,363 (each specifically incorporated herein by reference in its entirety). Solutions of the active compounds as freebase or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial ad antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, and the general safety and purity standards as required by FDA Office of Biologics standards.

Sterile injectable solutions are prepared by incorporating the disclosed pluralities of polymeric nanoparticulates and network nanoparticulate compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The compositions disclosed herein may be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically-acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared. If needed, the preparations may be further adapted for administration, as needed.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Synthesis of HPC Nanoparticles

HPC hydrogel nanoparticles were synthesized using an emulsion method. A 0.1 wt % HPC aqueous solution was prepared as follows: 0.1 g HPC powder was dispersed in 99.9 g aqueous sodium hydroxide solution (pH=12) by gentle stirring for a period of 4-6 days until HPC powder was thoroughly hydrolyzed. 0.475 g of dodecyltrimethylammonium bromide (DTAB) was added to 100 g of the 0.1 wt % HPC solution and the solution was stirred for 60 minutes. Then 0.04 g of divinylsulfone (DVS) used as a crosslinker was added to the HPC solution. After mixing completely, the solution was heated to about 55° C. Shortly thereafter, the color of the HPC solution changed to light blue indicating the formation of nanoparticles. The reaction was carried out for one hour at about 55° C. The resultant nanoparticles were dialyzed at least four times to remove the surfactant and NaOH. The same procedure was used to prepare the nanoparticles at 0.15 wt %, 0.3 wt %, and 0.5 wt % HPC solutions using different surfactant concentrations and different reaction temperatures, respectively.

FIG. 2 shows the hydrodynamic radius distributions (f(Rₕ)) of HPC nanoparticles (C=8.94×10⁻⁶ g/ml) in deionized water at 25° C. These particles were prepared in 0.1 wt % HPC solution at various DTAB concentrations. FIG. 3 shows the hydrodynamic radius distributions (f(Rₕ)) of HPC microgel particles (C=8.94×10⁻⁶ g/ml) in deionized water at 25° C. These particles were prepared with various HPC concentrations at 1 CMC of DTAB and at the reaction temperature of 55° C. FIG. 4 shows the hydrodynamic radii (Rₕ) at different reaction temperatures at 0.1 wt % HPC concentration and 1.54×10⁻² mol/l DTAB concentration.

### Example 2

### Synthesis of HPC Nanoparticle Networks

20 g of a 0.5 wt % HPC nanoparticle suspension were dispersed into an aqueous solution of sodium hydroxide at pH=12 and at a temperature of 55° C. Then, 0.02 ml of divinylsulfone (DVS) were added and the residual hydroxyl groups on the surfaces of neighboring HPC nanoparticles were bonded together to form a network. FIG. 1C shows an optical microscopic image of a HPC nanoparticle network in water at room temperature. The white bar represents 10 μ.

### Example 3

### Synthesis of Methacrylated Hydroxypropylcellulose

In a typical synthesis, 5.0 g of hydroxypropylcellulose (MW=30,000 g/mole) is dissolved into 200 mL of dry dimethylacetamide. Next, 1.1 g of methacryloyl chloride is added to the solution. The reaction is allowed to stir for 48 h. After this time, the solution is poured into 1 L of cold diethyl ether to precipitate the polymer. The collected polymer is then reprecipitated three times from 50 mL of hot acetonitrile. The polymer is placed under vacuum to remove excess solvent. ¹H-NMR is used to measure the amount of methacrylation of the polymer by integrating the geminal protons of the metharcylate group relative to the methyl protons of the isopropyl units on the polymer chain. The general reaction is illustrated in Scheme 1.

### Example 4

### Synthesis of Hydroxypropylcellulose Nanoparticles from Methacrylated Hydroxypropylcellulose. A Non-Surfactant Method

Into 400 mL of deionized water is dissolved 1.33 g of 5% methacrylated hydroxypropyl cellulose (polymer concentration of 0.33 wt. %). After the polymer has completely dissolved, the solution is purged with an inert gas (nitrogen or argon) for 20 minutes. The solution is then warmed to a temperature above the lower critical solution temperature of the solution (45° C. to 65° C.). Next, 4 mg of sodium metabisulfite followed by 4 mg of potassium persulfate are added to initiate the reaction. The reaction is stirred at the selected temperature for 20 minutes. After this time, the polymer is concentrated to about 2.5 wt. % by ultracentrifugation and redispersion. The particle size is measured by DLS using a NICOMP Model 370 Micron Particle Sizer. FIG. 8A and FIG. 8B show the dependence of size and population distributions with temperature.

### Example 5

### Synthesis of Degradable Crosslinker Used for Hydroxypropylcellulose Nanoparticle Formation

In a typical reaction, a 1 mole percent excess of hydroxyethylmethacrylate (1) is reacted with 2-bromo acetyl bromide (2) in chloroform. Excess potassium carbonate is used for base. The reaction proceeds in near quantitative yields in 16 h. Without purification the formed ester product (3) is reacted with excess succinic acid and sodium succinate in dimethylacetamide at 95° C. for 3 h. The reaction is then cooled to room temperature and poured into deionized water. The product (4) separates from water and is collected. After additional extractions with fresh deionized water, the product is taken up in chloroform and dried over MgSO₄. The chloroform is then removed under reduced pressure to yield a viscous oil with a slight yellow color. The general reaction is illustrated in Scheme 2.

### Example 6

### Synthesis of Degradable Crosslinker Functionalized Hydroxypropylcellulose

The general reaction is shown in Scheme 3. Typically, the portion of crosslinker needed for a particular functionalization level of the hydroxypropylcellulose is reacted with excess oxalyl chloride in chloroform for 3 h. Ratio of crosslinker to chloroform is about 2 g to 25 mL. After this time the chloroform is removed under reduced pressure to yield the acid chloride derivative of the degradable crosslinker. The acid chloride is then added to a hydroxypropylcellulose solution similar to that of Example 1. Purification and characterization follows that of Example 1.

### Example 7

### Loading of Fluorescein Labeled Bovine Serum Albumin (FITC BSA) into Hydroxypropylcellulose Nanoparticles

This example is similar to that of Example 4. Hydroxypropylcellulose functionalized with either degradable crosslinker or methacrylate side-groups is dissolved into deionized water to a concentration of 0.33 wt. %. The reaction is purged for 20 minutes with and inert gas. Next, fluorescein-labeled bovine serum albumin is added to the solution so that the total protein added is 5% wt/wt relative to hydroxypropylcellulose. The solution is warmed to above the lower critical solution temperature of the solution (45° C. to 65° C.). Sodium metabisulfite and potassium persulfate are added to the solution (0.2 to 0.4 wt % relative to polymer). The reaction is allowed to proceed for twenty minutes then cooled to room temperature.

### Example 8

### Formation of Hydroxypropylcellulose Nanoparticle Networks Using Nanoparticles Formed from the Non-Surfactant Method

Nanoparticles formed using the non-surfactant method containing either degradable crosslinker side-groups or the methacrylate side-groups have residual polymerizable groups. These groups are used to form the networks. Typically, 15 g of a suspension of nanoparticles in water (˜2.5 wt. %), is weighed into a 25-mL ultracentrifuge tube. The suspension is purged with an inert gas for 5 minutes. About 1.5 mg of sodium metabisulfite and 1.5 mg of potassium persulfate are added to the suspension. The suspension is agitated to dissolve the initiator and accelerator. The sample is then centrifuged at 35,000 rpm for 20 minutes using a Beckman LE-80 ultracentrifuge. The plug formed at the bottom of the tube is allowed to sit overnight before removal. For BSA loading, an amount of protein is loaded to correspond to 5 wt. % of the mass of the nanoparticles before initiation and plug formation. Determination of BSA loading is accomplished by analysis of the supernatant.

### Example 9

### Preparation of Hydroxypropylcellulose Nanoparticle Networks Containing FITC BSA in the Secondary Network Space

Nanoparticles formed using the non-surfactant method containing either degradable crosslinker side-groups or the methacrylate side-groups have residual polymerizable groups. These groups are used to form the networks. Typically, 15 g of a suspension of nanoparticles in water (˜2.5 wt %), is weighed into a 25-mL ultracentrifuge tube. The suspension is purged with an inert gas for 5 minutes. Next, about 18 mg of the BSA is added to the suspension and the suspension is agitated to dissolve the BSA. About 1.5 mg of sodium metabisulfite and 1.5 mg of potassium persulfate are added to the suspension. The suspension is agitated to dissolve the initiator and accelerator. The sample is then centrifuged at 25,000 rpm for 30 minutes using a Beckman LE-80 ultracentrifuge. The plug formed at the bottom of the tube is allowed to sit overnight before removal. UV-Vis analysis of the supernatant allows for the determination of the amount of BSA loaded into the network.

### Example 10

### Release Study of BSA from Hydroxypropylcellulose Networks

Typically, a mass of fully-hydrated network (30-150 mg) of known BSA content is rinsed 5 times with 100 mL portions of phosphate-buffered saline (pH 7.4) warmed to 37° C. This removes any surface BSA present on the network sample. The sample is then placed into a 20-mL scintillation vial containing 10 mL of phosphate buffered saline. This sample is incubated at 37° C. for the duration of the study. Aliquots are removed periodically for UV-Vis analysis. All aliquots are placed back into the sample container after analysis.

### Example 11

### Controlled Release of Small and Large Biomolecules from HPC Nanoparticle Network at the Same Time

5% methacrylated HPC polymer and 8 mg sodium metabisulfate as a initiator were added into 135 ml deionized-distilled water under nitrogen gas. At 43.5° C., just above the HPC phase transition, 8 mg potassium persulfate (KPS) was added. After 1 min., 15 ml BCG solution of 10 ppm was added. The reaction was carried on for 30 min. The HPC nanoparticles were formed with BCG entrapped. The colloidal dispersion was put on an ultracentrifuge with 30,000 rpm for 40 min to collect nanoparticles.

In the second step, the nanoparticles were re-dispersed and mixed with 25 ml water, 10 g of 50 ppm BSA, 5 mg sodium metabisulfate and 5 mg KPS. The dispersion was ultracentrifuged at 30,000 rpm for 40 min. After 18 h at room temperature, the nanoparticle network was formed with the BSA entrapped between the particles.

The HPC nanoparticle network that contains BCG in its nanoparticles and BSA between particles was immersed in a PBS buffer solution with pH=7.4 at 37° C. The time dependent drug release was monitored by a UV/Vis spectroscopy. The characterization absorptions for the BSA and the BCG were at 496 nm and 620 nm, respectively. As shown in FIG. 9, the HPC nanoparticle network could simultaneously release a small molecule (BCG) and a large molecule (BSA).

### Example 12

### UV-Vis Experiment of HRP Release and Activity

Loading of horseradish peroxidase (HRP) enzyme into HPC nanoparticle networks was performed as follows: An ultracentrifuge tube was charged with 1.08 mL of methacrylated HPC nanoparticles in MilliQ™ H₂O for a total dry polymer mass of 25 mg, 2.5 mg of horseradish peroxidase (235 U/mg) was added to the tube. The tube was capped with a septum and stirred. The solution was purged with N2(g) for 10 minutes, and 1.5 mg of potassium persulfate and 3.0 mg of sodium persulfate were added to the tube. The tube was centrifuged at 50,000 rpm for 30 min at room temperature and then allowed to sit at room temperature for 24 hours. The resulting nanoparticle network was dark brown in color and had a mass of 211 mg. Analysis of the supernatant indicated that the total mass of HRP in the particle was 0.51 mg indicating a loading efficiency of 20% for the enzyme.

Release of horseradish peroxidase from HPC NP network was performed as follows: A 100-mg fragment of HRP loaded HPC nanoparticle network was placed into 2.0 mL of PBS and the release of HRP was monitored by analysis of the supernatant with UV-Visible absorption. HRP has an absorption band at 403 nm. FIG. 13 shows the release of HRP from the HPC network over time.

Enzyme activity for HRP was determined using the production of quinoneimine from phenol and 4-aminoantipyrine in the presence of HRP and hydrogen peroxide and was monitored using UV-Visible absorption spectroscopy. Release of horseradish peroxidase from HPC NP network: A 25-mg fragment of HRP loaded HPC nanoparticle network was placed into 500 mL of PBS and the release of HRP was monitored by analysis of the supernatant with UV-Visible absorption. 1-mL aliquots were removed and diluted with 2 mL of a solution containing 32.4 mg phenol and 1.0 mg of 4 aminoantipyrine. 1.0 mL of 0.003% hydrogen peroxide solution was added to an aliquot and the solution studied by UV-visible at 0 and 5 minutes. The activity of the free enzyme was found to be 0.00351 units/mg (quinoneimine)·min and the activity of the enzyme at 0, 1, 3, 5, 8 and 24 hours of release from the network was found to be 98% of this or better as seen in the following table:

| | Activity |
|---|---|
| Time (hr) | Units/mg · min |
| 0 | 0 |
| 1 | 0.00341 |
| 3 | 0.00336 |
| 5 | 0.00334 |
| 8 | 0.00335 |
| 24 | 0.00330 |

A 25-mg fragment of HRP loaded HCP nanoparticle network was immersed in 500 mL of a solution containing 1.0 mg/mL trypsin, 4.0 mg/mL CaCl₂, in phosphate buffered saline (pH=7.4) at 37° C. 1.0 mL aliquots were removed at 0, 5, and 30 minutes and the concentration and activity of the released HRP assayed as shown in Example 12. The activity of the free HRP is 0.0035 Units/mg·min while the HRP released in the presence of trypsin is severely compromised as shown in the following table.

| | Activity |
|---|---|
| Time (hr) | Units/mg · min |
| 0 | 0 |
| 1 hr | 0.003326 |
| 5 hr | 0.003337 |
| 8 hr | 0.003321 |

The network was removed from the trypsin containing buffer and washed with a copious amount of PBS. The network was immersed in 500 mL of PBS and assayed for activity as shown in Example 12. As shown in the following table, the activity of the enzyme remaining in the network was at least 95% of the free HRP at 0.0035 Units/mg·min.

### Example 13

### Synthesis of Biodegradable NIPA Nanoparticles

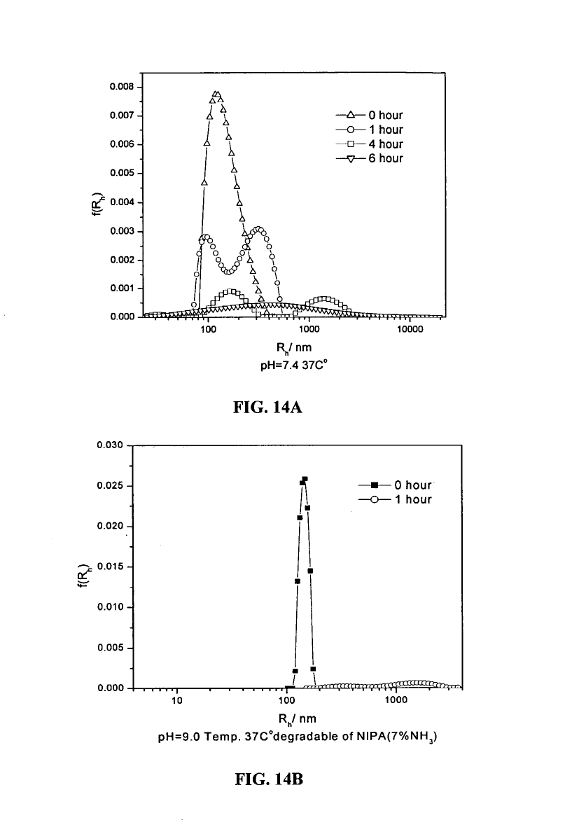

The degradable NIPA nanoparticles were formed at pH=5.4 buffer solution with 1.62 g NIPA monomer, 0.165 g 3-amimopropyl methacrylamide (7% mm), and 0.06 g biodegradable crosslinker at 53° C. DTAB was 0.056 g and KPS as an initiator was 50 mg. After 2 h, the particles were formed. The NIPA particle was exhaustively dialyzed in a dialysis tube for 7 days at 4° C. The deionized water out of the tube was changed three times a day. FIG. 14 shows the degradation of degradable NIPA particles at different pH values. The degradation rate at pH=7.4 was slower than that at pH=9.0.

### Example 14

### Release of BCG from NIPA Nanoparticles

The entrapment of bromocresol green in NIPA nanoparticles with degradable and non-degradable crosslinkers and subsequent release of bromocresol green from nanoparticles was performed as follows:

| | Activity |
|---|---|
| Time (min) | Units/mg · min |
| 0 | 0 |
| 5 min | 0.000012 |
| 30 min | 0.0000043 |

A) Non-degradable: A 500-mL media bottle equipped with a stir bar was charged with 3.78 g (33.5 mmol)of N-isopropylacrylamide, 66 mg (0.43 mmol) of methylene bisacrylamide, 0.015 g (0.052 mmol) sodium dodecyl sulfate, 1.82 mg (0.0026 mmol) of bromocresol green dye, and 240 mL of 10 mmol/L acetic acid/sodium acetate buffer (pH=5.2) in milli Q H20. The flask was capped and purged with N2(g) for 1 hr while stirring. 0.166 g (0.61 mmol) of K₂S₂O₈ was dissolved into 21 mL of MilliQ™ H20 and injected into the monomer solution. The solution was transferred to a 40° C. water bath for 6 hrs. The resulting nanoparticles were purified by repeated ultracentrifuge and flushing with acetate buffer solution. Light scattering analysis indicated that the particles were approximately 400 nm in diameter. The solution had a green tint in acetate buffer due to the entrapped dye molecules. Analysis of the supernatant indicated that the dye loading efficiency was 14%.

B) Degradable: Each of 8 500-mL media bottles equipped with 1-inch stir bars were charged with 3.78 g (33.5 mmol) of N-isopropylacrylamide, 0.015 g (0.052 mmol) sodium dodecyl sulfate, and 1.82 mg (0.0026 mmol) of bromocresol green dye. The following masses of degradable crosslinker were added to each reaction bottle respectively:

| HEAmGly)₂Suc | | | | HEAmLac)₂Suc | | | |
|---|---|---|---|---|---|---|---|
| 92.1 | 184 | 276 | 368 | 97.7 | 195 | 293 | 391 |
| mg | mg | mg | mg | mg | mg | mg | mg |
| 0.35 | 0.42 | 0.67 | 1.0 | 0.35 | 0.42 | 0.67 | 1.0 |
| mmol | mmol | mmol | mmol | mmol | mmol | mmol | mmol |

240 mL of 10 mmol/L acetic acid/sodium acetate buffer (pH=5.2) in MilliQ™ H₂O was added to each bottle. The flask was capped and purged with N2(g) for 1 hr while stirring. 0.166 g (0.61 mmol) of K₂S₂O₈ was dissolved into 21 mL of MilliQ™ H₂O and injected into the monomer solution. The solution was transferred to a 40° C. water bath for 6 hrs. The resulting nanoparticles were purified by repeated ultracentrifuge and flushing with acetate buffer solution. Light scattering analysis indicated that the particles were approximately 520 nm in diameter. The solution had a green tint in acetate buffer due to the entrapped dye molecules. Analysis of the supernatant indicated that the dye loading efficiency was 9%.

The release of trapped bromocresol green dye was studied by dissolving 0.5 g of hydrated nanoparticles into 100 mL of phosphate buffered saline (pH=7.4) at 37° C. Aliquots were pulled and filtered using a 10000 molecular weight cutoff centrifugal ultrafiltration device and the permeate analyzed for bromocresol green dye release by UV-Visible absorption. FIG. 14 shows the release of bromocresol green over time for varying crosslinker compositions and concentrations.

### Example 15

### Synthesis of NIPA Nanoparticle Network

3.79 g NIPA monomer, 0.099 g AA monomer, 66 mg methylene bis-acrylamide as a crosslinker, 0.116 g sodium dodecylsulfate as a surfactant, and 240 ml deionized water were mixed in a reactor. The solution was heated to 70° C. and purged under nitrogen for 40 min. Then a solution containing 0.166 g of potassium persulfate dissolved in 21 ml of deionized water was added to initiate the polymerization reaction. The reaction was carried out at 70° C. for 4.5 h.

### Example 16

### Synthesis of Co-Nanoparticle Networks

The HPC particles were formed at pH=12.00. 0.5% HPC (MW=100,000) water solution of 100 g was mixed with 1.27 g DTAB, and DVS(divinylsulfone) 0.1 g as a crosslinker at 68° C. After adding DVS for 40 min, the HPC particles were formed. The HPC particles were exhaustively dialyzed in a dialysis tube for 7 days. The deionized water out of the tube was changed three times a day.

The PVA nanoparticles were prepared using a surfactant-free method. PVA (88 mol % hydrolyzed, MW ˜25,000, Polysciences, Inc.) was dissolved in distilled water. Sodium hydroxide solution (5 M) was added to yield 0.5 wt % PVA solution at pH=12. Then, about 56 g of acetone were added to 100 g of PVA solution. After stirring about 30 min, 0.1 g DVS was added to the solution. The reaction lasted about six hours and the resulting nanoparticle dispersion was dialyzed for 7 days. The deionized water out of the tube was changed three times a day.

The hydrodynamic radius distributions of HPC and PVA particles are shown in FIG. 17A.

HPA and PVA dispersions were then condensed to 5 wt %. Different amounts of the PVA and HPC nanoparticles were then mixed. There were 5 different samples: homo HPC, 2:1 HPC:PVA co-nanoparticle network, 1:1 HPC:PVA, 1:2 HPC:PVA, and homo PVA nanoparticle network. Crosslinking was performed by adding divinylsulfone to the PVA-HPC mixed dispersion at pH=12 at room temperature. The PVA-HPC co-nanoparticle network was formed within 1 hour.

Temperature dependent swelling ratios of the HPC-PVA co-nanoparticle networks are shown in FIG. 17B. It can be seen from the figure that the HPC nanoparticle network has the lowest LCST, while PVA exhibit no temperature responsive property. As PVA nanoparticle concentration increases, the phase transition temperature increases.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the composition, methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims. Accordingly, the exclusive rights sought to be patented are as described in the claims below:

The documents and references cited in this application are incorporated by reference herein.

U.S. PATENT DOCUMENTS

| | | | |
|---|---|---|---|
| Re35068 | October 1995 | Tanaka et al. | 523/300. |
| 4,732,930 | March 1988 | Tanaka et al. | 524/742. |
| 5,100,933 | March 1992 | Tanaka et al. | 523/300. |
| 5,183,879 | February 1993 | Yuasa et al. | 528/503. |
| 5,403,893 | April 1995 | Tanaka et al. | 525/218. |
| 5,532,006 | July 1996 | Lauterber et al. | 424/9.322 |
| 5,580,929 | December 1996 | Tanaka et al. | 525/218. |
| 6,030,442 | February 2000 | Kabra, et al. | 536/84 |
| 4,912,032 | March 1990 | Hoffman et al. | 435/7.1 |
| 6,194,073 | February 2001 | Li, et al. | 428/420 |
| 5,976,648 | November 1999 | Li, et al. | 428/34. |
| 5,062,841 | November 1991 | Siegel | 604/891.1 |
| 5,654,006 | August 1997 | Fernandez, et al. | 424/489 |
| 6,030,442 | February 2000 | Kabra, et al. | 106/162.8 |
| 6,187,599 | February 2001 | Asher, et al. | 436/531 |
| 4,555,344 | November 1985 | Cussler | |

FOREIGN PATENT DOCUMENTS

| | | |
|---|---|---|
| 0 365 011 A2 | April 1990 | EP |
| 2-155952 | June 1990 | JP |
| 3-701 | January 1991 | JP |
| 7-82325 | March 1995 | JP |
| 7-292040 | November 1995 | JP |
| WO 91/05816 A1 | May 1991 | WO |
| WO 92/02005 A2 | February 1992 | WO |
| WO 95/31498 A1 | November 1995 | WO |

### Other References

Peppas, N. A., Hydrogels in Medicine and Pharmacy, (CRC Press, Boca Raton, Fla., 1987).

Peppas, N. A. et al., “New challenges in biomaterials,” Science 263, 1715 (1994).

Tanaka, T., “Collapse of gels and the critical endpoint,” Phys. Rev. Lett. 40, 820-823 (1978).

Tanaka, T., et al. “Collapse of gels in an electric field,” Science 218, 467 (1982).

Osada, Y. et al., “A polymer gel with electrically driven mobility,” Nature 355, 242 (1992);

Osada, Y., et al., “Intelligent gels,” Scie. Ame. 268, 82-87 (1993).

Siegel, R. A., et al., “pH-dependent equilibrium swelling properties of hydrophobic polyelectrolyte copolymer gels,” Macromol. 21, 3254-3259 (1988).

Chen, et al., “Graft copolymer compositions that exhibit temperature-induced transitions over a wide range of pH,” Nature 373, 49 (1995).

Yeong, B., et al., “Biodegradable block copolymers as injectable drug-delivery systems,” Nature 388, 860-863 (1997).

Kiser, P. F., et al. “A synthetic mimic of the secretory granule for drug delivery,” Nature 394, 459 (1998).

Weissman, J. M., et al., “Thermally switchable periodicities and diffraction from mesoscopically ordered materials,” Science 274, 959-960 (1996).

Holtz, J. H., et al. “Polymerized colloidal crystal hydrogel films as intelligent chemical sensing materials,” Nature 389, 829-832 (1997).

Hu, Z, et al., “Synthesis and application of modulated polymer gels,” Science 269, 525-527 (1995).

Hu, Z., et al. “Polymer gels with engineered environmentally-responsive surface patterns,” Nature 393, 149-(1998).

Frank, S., et al. “Voltage-sensitive magnetic gels as magnetic resonance monitoring agents” Nature 363, 334 (1993).

Hu, Z, et al., “Polymer gel nanoparticle networks,” Advanced Materials Advanced Materials 12, 1173 (2000).

Lu, et al., “Synthesis and light scattering study of hydroxypropyl cellulose microgel,” Macromolecules, Nov. 14 (2000).

Kabra, B. G., et al., Macromolecules 1998, 31, 2166.

Harsh, D. C. et al., J. Controlled Release 1991, 17, 175.

Pelton, R. H., et al., “Preparation of Aqueous Latices with N-Isopropylacrylamide.” Colloids and

Surfaces 20, 247-256 (1986).

Li, Y., et al. J. Chem. Phys. 1990, 92, 1365.

Kabra, B. G., Polymer Comm. 1991, 32, 322.

Yoshida, R., et al., Nature 1995, 374, 240.

Hirotsu, S., et al., J. Chem. Phys. 1987, 87, 1392.

Kamenetzky, E. A., et al., Science 1994, 263, 207.

What is claimed is:

1. A nanostructured polymeric material comprising environmentally responsive nanoparticles, alone or in combination with non-environmentally responsive nanoparticles, that are bonded through functional groups on surfaces of neighboring particles to form a network.

2. The nanostructured polymeric material of claim 1, wherein the network comprises two levels of structural difference; a primary network comprised of crosslinked polymer chains inside each nanoparticle and a secondary network comprising of a crosslinked system of the nanoparticles themselves.

3. The nanostructured polymeric material of claim 2, wherein mesh size of the primary network is between 1-10 nm and the mesh size of the secondary network is between 50-500 nm.

4. The nanostructured polymeric material of claim 1 in which changes in the physical properties of the environmentally responsive nanoparticles such as hydrophobicity, volume change, and elasticity are brought about by changes in environmental conditions, including pH, temperature, electric current, ionic strength, type of solvent and pressure.

5. The nanostructured polymeric material of claim 1 further comprising N-isopropyl acrylamide or related analog, Hydroxypropylcellulose or related analog, polyvinyl alcohol or other analog, polypropylene oxide or related analog, polyethylene oxide, or related analog, polyethylene oxide/polypropylene oxide copolymers, or other known environmentally responsive polymer, alone or in combination thereof.

6. The nanostructured polymeric material of claim 1 further comprising nanoparticles in a particle size range of 1-1000 nanometers in diameter.

7. The nanostructured polymeric material of claim 1 further comprising nanoparticles in a particle size range of 1-1000 nanometers in diameter particle size range of 1-500 nanometers.

8. The nanostructured polymeric material of claim 1 further comprising nanoparticles in a particle size range of 1-1000 nanometers in diameter particle size range of 20-500 nanometers.

9. The nanostructured polymeric material of claim 1 comprising nanoparticles that are internally crosslinked with non-degradable or degradable crosslinking compounds and bonded through functional groups on surfaces of neighboring particles with non-degradable or degradable crosslinking compounds to form a network.

10. The material of claim 9 where the degradable crosslinking compounds have a monomeric or oligomeric composition comprising a polyacid with at least two acidic groups directly or indirectly connected to reactive groups usable to cross-link polymer filaments, wherein between at least one reactive group and the polyacid is a degradable sequence consisting of a hydroxyalkyl acid ester sequence having a number of hydroxyalkyl acid ester groups selected from the group consisting of 1, 2, 3, 4, 5 and 6; the cross-linker being usable to form cross-linked polymer filaments with defined degradation rates.

11. The nanostructured polymeric materials of claim 9 further containing at least one pharmaceutically active compound.

12. The material of claim 11 wherein said pharmaceutically active compound resides inside individual nanoparticles, between the nanoparticles or in both domains.

13. The nanostructured polymeric material of claim 11 used as a drug delivery device in vivo to treat a variety of wounds, diseases and disorders in humans and mammals.

14. The nanostructured polymeric material of claim 13 capable of providing a variety of controlled release rates of pharmaceutically active compounds through variations in the particle size of the nanoparticles composing the networks and/or biodegradable crosslinkers used in claim 13.

15. The nanostructured polymeric material of claim 11, wherein said pharmaceutically active compound or combination of pharmaceutically active compounds comprises an anti-allergic agent.

16. The nanostructured polymeric material of claim 15, wherein said anti-allergic agent is amlexanox, astemizole, azelastinep, emirolast, alopatadine, cromolyn, fenpiprane, repirinast, tranilast, or traxanox.

17. The nanostructured polymeric material of claim 11, wherein said pharmaceutically active compound or combination of pharmaceutically active compounds comprises an anti-inflammatory analgesic agent.

18. The nanostructured polymeric material of claim 17, wherein said anti-inflammatory analgesic agent is acetaminophen, methyl salicylate, monoglycol salicylate, aspirin, mefenamic acid, flufenamic acid, indomethacin, diclofenac, alclofenac, diclofenac sodium, ibuprofen, ketoprofen, naproxen, pranoprofen, fenoprofen, sulindac, fenclofenac, clidanac, flubiprofen, fentiazac, bufexarnac, piroxicam, phenylbutazone, oxyphenbutazone, clofezone, pentazocine, mepirizole, or tiaramide hydrochloride.

19. The nanostructured polymeric material of claim 11, wherein said pharmaceutically active compound or combination of pharmaceutically active compounds comprises an antianginal agent.

20. The nanostructured polymeric material of claim 19, wherein said antianginal agent is nifedipine, atenolol, bepridil, carazolol, or epanolol.

21. The nanostructured polymeric material of claim 11, wherein said pharmaceutically active compound or combination of pharmaceutically active compounds comprises a steroidal anti-inflammatory agent.

22. The nanostructured polymeric material of claim 21, wherein said steroidal anti-inflammatory agent is hydrocortisone acetate, predonisolone acetate, methylpredonisolone, dexamethasone acetate, betamethasone, betamethasone valerate, flutetasone, fluormetholone, or beclomethasone diproprionate.

23. The nanostructured polymeric material of claim 11, wherein said pharmaceutically active compound or combination of pharmaceutically active compounds comprises an antihistamine.

24. The nanostructured polymeric material of claim 23, wherein said antihistamine is diphenhydramine hydrochloride, chlorpheniramine maleate, isothipendyl hydrochloride, tripelennamine hydrochloride, promethazine hydrochloride, or methdilazine hydrochloride.

25. The nanostructured polymeric material of claim 11, wherein said pharmaceutically active compound or combination of pharmaceutically active compounds comprises a local anesthetic.

26. The nanostructured polymeric material of claim 25, wherein said local anesthetic is dibucaine hydrochloride, dibucaine, lidocaine hydrochloride, lidocaine, benzocaine, p-buthylaminobenzoic acid, 2-(di-ethylamino) ethyl ester hydrochloride, procaine hydrochloride, tetracaine, tetracaine hydrochloride, chloroprocaine hydrochloride, oxyprocaine hydrochloride, mepivacaine, cocaine hydrochloride, piperocaine hydrochloride, dyclonine, or dyclonine hydrochloride.

27. The nanostructured polymeric material of claim 11, wherein said pharmaceutically active compound or combination of pharmaceutically active compounds comprises a bactericide or disinfectant.

28. The nanostructured polymeric material of claim 27, wherein said bactericide or disinfectant is thimerosal, phenol, thymol, benzalkonium chloride, chlorhexidine, povidone iodine, cetylpyridinium chloride, eugenol, trimethylammonium bromide, benzoic acid or sodium benzoate.

29. The nanostructured polymeric material of claim 11, wherein said pharmaceutically active compound or combination of pharmaceutically active compounds comprises a vasoconstrictor.

30. The nanostructured polymeric material of claim 29, wherein said vasoconstrictor is naphazoline nitrate, tetrahydrozoline hydrochloride, oxymetazoline hydrochloride, phenylephrine hydrochloride, or tramazolinehydrochloride.

31. The nanostructured polymeric material of claim 11, wherein said pharmaceutically active compound or combination of pharmaceutically active compounds comprises a hemostatic agent.

32. The nanostructured polymeric material of claim 31, wherein said hemostatic agent is thrombin, phytonadione, protamine sulfate, aminocaproic acid, tranexamic acid, carbazochrome, carbaxochrome sodium sulfate, rutin, or hesperidin.

33. The nanostructured polymeric material of claim 11, wherein said pharmaceutically active compound or combination of pharmaceutically active compounds comprises a chemotherapeutic agent.

34. The nanostructured polymeric material of claim 33, wherein said chemotherapeutic agent is sulfamine, sulfathiazole, sulfadiazine, homosulfamine, sulfisoxazole, sulfisomidine, sulfamethizole, nitrofurazone, taxanes, platinum compounds, topoisomerase 1 inhibitors, or anthrocycline.

35. The nanostructured polymeric material of claim 11, wherein said pharmaceutically active compound or combination of pharmaceutically active compounds comprises an antibiotic.

36. The nanostructured polymeric material of claim 35, wherein said antibiotic is penicillin, meticillin, oxacillin, cefalotin, cefalordin, erythromycin, lincomycin, tetracycline, chlortetracycline, oxytetracycline, chloramphenicol, kanamycin, streptomycin, gentamicin, bacitracin, cycloserine, or clindamycin.

37. The nanostructured polymeric material of claim 1, wherein said pharmaceutically active compound or combination of pharmaceutically active compounds comprises a keratolytic agent.

38. The nanostructured polymeric material of claim 37, wherein said keratolytic agent is salicylic acid, podophyllum resin, podolifox, or cantharidin.

39. The nanostructured polymeric material of claim 11, wherein said pharmaceutically active compound or combination of pharmaceutically active compounds comprises a cauterizing agent.

40. The nanostructured polymeric material of claim 39, wherein said cauterizing agent is chloroacetic acid or silver nitrate.

41. The nanostructured polymeric material of claim 11, wherein said pharmaceutically active compound or combination of pharmaceutically active compounds comprises a hormone.

42. The nanostructured polymeric material of claim 41, wherein said hormone is estrone, estradiol, testosterone, equilin, or human growth hormone.

43. The nanostructured polymeric material of claim 11, wherein said pharmaceutically active compound or combination of pharmaceutically active compounds comprises a growth hormone inhibitor.

44. The nanostructured polymeric material of claim 43, wherein said growth hormone inhibitor is octreotide or somatostatin.

45. The nanostructured polymeric material of claim 11, wherein said pharmaceutically active compounds or combination of pharmaceutically active compounds comprises an analgesic narcotic.

46. The nanostructured polymeric material of claim 45, wherein said analgesic narcotic is fentanyl, buprenorphine, codeine sulfate, levophanol, or morphine hydrochloride.

47. The nanostructured polymeric material of claim 11, wherein said pharmaceutically active compound or combination of pharmaceutically active compounds comprises an antiviral drug.

48. The nanostructured polymeric material of claim 47, wherein said antiviral drugs are protease inhibitors, thymadine kinase inhibitors, sugar or glycoprotein synthesis inhibitors, structural protein synthesis inhibitors, attachment and adsorption inhibitors, and nucleoside analogues including acyclovir, penciclovir, valacyclovir, or ganciclovir.

49. The nanostructured polymeric material of claim 11, wherein the pharmaceutically active compound or combination of pharmaceutically active compounds is between about 0.001 and about 30 percent by weight of the material.

50. The nanostructured polymeric material of claim 11, wherein the pharmaceutically active compound or combination of pharmaceutically active compounds is between between about 0.005 and about 20 percent by weight of the material.

51. The nanostructured polymeric material of claim 1, wherein the bonding of the nanoparticles together contributes to the structural stability of the network, while packing arrangement and size of the nanoparticles provide structures that can diffract light.

52. The nanostructured polymeric material of claim 51, wherein said material is usable as an optical sensor using its environmentally responsive properties.

53. The nanostructured polymeric material of claims 9 containing a chemical agent or combination of chemical agents other than a pharmaceutically active compound.

54. The nanostructured polymeric material of claim 53, wherein the chemical agent is a pesticide, fungicide, fertilizer, or other agricultural material, a cationic, anionic, non-ionic exchange material or other complexing compound.

55. A composition comprising the nanostructured polymeric material of claim 1.

56. The composition of claim 55, formulated for bioremediation.

57. The composition of claim 55, formulated as a mucoadhesive or a bioadhesive.

58. The composition of claim 55, further comprising at least a first pharmaceutical excipient.

59. The composition of claim 58, formulated for administration to an animal.

60. The composition of claim 58, formulated for parental administration to an animal.

61. A controlled-release pharmaceutical delivery system comprising the composition of claim 55, and at least a first diagnostic, therapeutic, or prophylactic medicament.

62. The controlled-release pharmaceutical delivery system of claim 61, formulated for oral, intravenous, intraarterial, intradermal, subcutaneous, sublingual, inhalation, transdermal, intrathecal, intraossius, intranasal, intraocular, or intracellular administration.

63. A therapeutic kit comprising the nanostructured polymeric material of claim 1, the composition of claim 55, or the controlled-release pharmaceutical delivery system of claim 62, and instructions for using said kit.

64. The kit of claim 63, wherein said kit further comprises at least a first peptide, polypeptide, protein, vaccine, antisense oligonucleotide, hormone, growth factor, polynucleotide, vector, ribozyme, or at least a first diagnostic, therapeutic, or prophylactic medicament.

65. A method of controlling the delivery of a pharmaceutical compound to a target site, said method comprising providing to said site, the controlled-release pharmaceutical delivery system of claim 61, for a time effective to deliver said compound to said site.

66. A method of delaying or sustaining the delivery of a pharmaceutical compound to a first target site of a mammal, said method comprising administering to said mammal the controlled-release pharmaceutical delivery system of claim 61, in an amount and for a time effective to delay or sustain the delivery of said compound to said target site within said mammal.

67. The method of claim 66, wherein said target site is a cell, tissue, gland, bone, tumor, or an organ within the body of said mammal.

68. The method of claim 66, wherein said mammal is (a) a human, or (b) a non-human mammal under the care of a veterinarian.

69. The method of claim 66, wherein said compound is delivered to said target site within a period of from about 10 min to about 24 hrs following administration of said pharmaceutical delivery system to said mammal.

70. The method of claim 66, wherein said compound is delivered to said target site within a period of from about 20 min to about 12 hrs following administration of said pharmaceutical delivery system to said mammal.

71. The method of claim 66, wherein said compound is delivered to said target site within a period of from about 30 min to about 6 hrs following administration of said pharmaceutical delivery system to said mammal.

72. The method of claim 66, wherein said compound is delivered to said target site within a period of from about 1 hr to about 3 hrs following administration of said pharmaceutical delivery system to said mammal.

73. A method of remediating a contaminated site, comprising contacting said site with, or providing to said site, an amount of the composition of claim 55 effective to remediate said site.

74. The method of claim 73, wherein said site is an environmental, commercial, residential or industrial site, or the site of an industrial accident.

75. The method of claim 73, wherein said site comprises a radioactive, chemical, or biological contaminant.

76. The method of claim 73, wherein said composition comprises a nanoparticle network that comprises at least a first functionalized moiety, or a free ionic charge on at least a first surface of said nanoparticle or said nanoparticle network.

77. A bioadhesive material that comprises the composition of claim 57.

78. The bioadhesive material of claim 77, comprising nanoparticles that comprise at least a first polymer selected from the group consisting of HPC, NIPA, PVA, PPO, PEO, PPO copolymer, and PEO.

79. A method of preparing a nanostructured polymeric gel, comprising the steps of:
(a) contacting a plurality of polymeric gel nanoparticles under conditions effective to permit self-assembly of a substantial population of said polymeric gel nanoparticles into a network of nanoparticles; and
(b) reacting said network of nanoparticles with at least a first cross-linking agent under conditions effective to substantially covalently crosslink said network of nanoparticles to produce said nanostructured polymeric gel.

80. The method of claim 79, wherein said crosslinking agent is a degradable crosslinking agent.

81. The method of claim 79, wherein said crosslinking agent is a biodegradable crosslinking agent.

82. The method of claim 79, wherein said crosslinking agent is divinyl sulfone.

83. The method of claim 79, wherein said plurality of polymeric gel nanoparticles comprises HPC, NIPA, PVA, PPO, PEO, PPO copolymer, or PEO copolymer nanoparticles.

84. The method of claim 79, wherein said plurality of polymeric gel nanoparticles comprises HPC.

85. The method of claim 79, wherein said plurality of polymeric gel nanoparticles comprises a population of internally-crosslinked nanoparticles.

86. The method of claim 79, wherein said plurality of polymeric gel nanoparticles comprises a population of nanoparticles internally-crosslinked using a non-degradable crosslinking agent.

87. The method of claim 79, wherein said plurality of polymeric gel nanoparticles comprises a population of colloidal nanoparticles.

88. The method of claim 79, wherein said plurality of polymeric gel nanoparticles are prepared by precipitation.

89. The method of claim 79, wherein said plurality of polymeric gel nanoparticles are prepared by precipitation from a solution that comprises at least a first surfactant.

90. The method of claim 89, wherein said at least a first surfactant comprises DTAB.

91. The method of claim 89, wherein said plurality of polymeric gel nanoparticles have an average particle size of from about 1 to about 5000 nm.

92. The method of claim 79, wherein said plurality of polymeric gel nanoparticles have an average particle size of from about 5 to about 2000 nm.

93. The method of claim 79, wherein said plurality of polymeric gel nanoparticles have an average particle size of from about 10 to about 1000 nm.

94. The method of claim 79, wherein said plurality of polymeric gel nanoparticles have an average particle size of from about 50 to about 500 nm.
